(19) United States
(12) Patent Application Publication
(10) Pub. No.: US 2004/0161756 A1
(43) Pub. Date: Aug. 19, 2004

(54) Substrate linked directed evolution (slide)

(76) Inventors: Adrian Francis Stewart, Dresden (DE); Frank Buchholz, San Francisco, CA (US)

Correspondence Address: WENDEROTH, LIND & PONACK, L.L.P., 2033 K STREET N. W., SUITE 800, WASHINGTON, DC, 20006-1021, US

(21) Appl. No.: 10/433,311

(22) PCT Filed: Nov. 30, 2001
(86) PCT No.: PCT/IB01/02787

(30) Foreign Application Priority Data
Dec. 1, 2000 (GB) 0029375.3

(51) Int. Cl. C12Q 1/68

(52) U.S. Cl. 435/6

(57) ABSTRACT

The invention relates to methods for the evolution of molecules with improved biological properties. In particular, the invention relates to methods using proteins that modify a DNA substrate to establish a link between the action of these proteins and the selection of molecules with improved biological properties. A unifying feature of all these mechanisms is that the coding region being evolved is in the same genetic element or on the same DNA molecule as a target site for a DNA-modifying protein. Accordingly, the activity (or inactivity) of the DNA-modifying protein can be tested by evaluating the sequence of its nucleic acid substrate. In this manner, a number of different types of compounds may be selected, including improved DNA-modifying proteins, improved substrates for DNA-modifying proteins, improved ligand-receptor interactions, improved co-factor and regulatory protein activities, improved DNA-binding proteins, and so on.

The present invention relates to methods for the evolution of molecules with improved biological properties. In particular, the invention relates to methods using proteins that act on DNA to establish a link between the action of these proteins and the selection of molecules with improved biological properties.

All documents cited herein are hereby incorporated by reference.

Directed in vitro evolution is a powerful method for the generation of molecules that possess desired biological properties. In this method, the key processes of Darwinian evolution, namely random mutagenesis, recombination and selection, are mimicked in vitro in order to evolve molecules with new or improved biological properties.

A number of different approaches have conventionally been taken to generate novel polypeptides with new, modified, or improved biological activity. For molecules of known structure, these methods have involved the directed alteration of residues in specific areas of the molecule (Winter et al., 1982). In the absence of structural information, genetic diversity for directed protein evolution has primarily been generated by point mutagenesis, combinatorial cassette mutagenesis (Black et al., 1996) or by DNA shuffling (Stemmer et al., 1994). Novel molecules have also been generated by phage display (Marks et al., 1994).

One problem with mimicking evolution by any method that utilises sequential random mutagenesis is that deleterious mutations appear simultaneously with beneficial mutations and become fixed, such that the evolutionary potential of the method becomes limited. Additionally, many beneficial mutations are discarded in the selection step, since only the mutation chosen to parent the next generation is retained.

Furthermore, the fact that the genetic element that encodes the molecule with the desired biological activity is not encoded in the same molecule as that selected for means that recovery of the genetic code is a difficult and time-consuming task. The problem of protein evolution relates to the separation of informational and functional components. The informational molecule (DNA or RNA) that encodes the favourable mutation(s) does not itself convey the improved biological property, rather, this is conveyed by the corresponding protein translated from the encoded information.

Protein evolution strategies are therefore constrained by the necessity to maintain a physical relationship between the favourable mutation(s) and the improved property. Usually this has been accomplished by association within a compartment provided by a host cell or phage where both the gene encoding the favourable protein and the protein itself are entrapped together. Consequently, most protein evolution exercises performed to date require maintenance of the integrity of the host during the screen for the improved biological property through steps to isolate the successful candidate before retrieval of the informational molecule. This requirement imposes limitations on the evolutionary cycle employed both in terms of cycle speed and scale.

Two alternative molecular evolution approaches have been described that link the informational and functional components in different ways. Both simplify aspects of the molecular evolution cycle and deliver advantages in terms of speed and scale. In certain in vitro RNA or DNA evolution exercises, the informational and functional components are carried by the same molecule; linkage by compartmentalisation is thus not required (Beaudry and Joyce (1992) 257:635-641; Lehman and Joyce (1993) Nature 361:182-185; Wright and Joyce (1997) Science 276: 614-617; Breaker and Joyce (1994) Chem Biol 1:223-229).

In the particular case of molecular evolution based on ribozymes, the same RNA molecule provides the template that encodes the enzyme, the enzyme itself and substrate upon which the enzyme acts. Hence selection for improved enzyme activity concomitantly delivers the molecule encoding the improved enzyme. These examples do not involve molecular evolution of protein since the enzyme may only be a nucleic acid molecule.

A second approach involves the incorporation of the antibiotic puromycin into an RNA molecule encoding the protein (Roberts and Szostak (1997) P.N.A.S. USA 94:12297-12302). After translation, the protein and RNA molecules are covalently linked through the puromycin moiety. Hence the informational and functional components are physically linked and compartmentalisation is not required. Although the approach relieves from some of the disadvantages of compartmentalisation, an additional step is required to convert the informational molecule from RNA to DNA for amplification.

For the selection of enzymes, a number of drawbacks exist, meaning that the generation of novel or improved enzymes has proven difficult. The main obstacles result from a paucity of methods for selection; although it is simple to select for catalytic activity, the selection of the genetic code itself is difficult, since in methods proposed to date, there is no direct connection between phenotype and genotype.

Initial attempts to improve enzyme properties by mimicking the natural process of evolution used mutant microorganisms, selecting for increased enzyme activity by way of growth advantage (Cunningham and Wells, 1987). More recently, phages displaying catalytic molecules have been enriched by binding to suicide inhibitors that bind irreversibly to the protein (Soumillion et al., 1994). However, suicide inhibitors or transition state analogues are not generally available for every reaction of interest. A direct selection for the desired catalytic activity would yield better results.

To generate molecules with improved binding characteristics, most conventional methods have relied on iterative steps of mutagenesis and screening, whereby molecules possessing desirable properties are selected by virtue of their affinity for target. In addition to those mentioned above, specific problems in this area of molecule design are that the efficiency of the selection process limits its effectiveness in producing molecules with high affinity for target. Furthermore, limitations on library size reduce the possible number of mutations that can be screened.

In most cases of protein molecular evolution described to date, the gene encoding the protein of interest has been randomly mutated to create a library of candidate molecules. However the theoretical number of mutant variations of any given protein is vast and greatly exceeds the practical limits imposed by current approaches for screening mutant libraries. Although (i) current methodologies permit the creation of very large mutant libraries; and (ii) the chances that a library contains a favourable mutant combination increases with the size of the library, the practical limits imposed by current approaches for screening mutant libraries restricts the practice. Hence any approach that addresses these practical limitations so that larger libraries can be screened will improve the current art.

The practical restrictions on library screening imposes two further limitations on applications of molecular evolution. Current approaches rely on selection of mutant candidates that are clearly favourable under the selection criterion applied. These favourable mutants are then used to seed the next round of library construction and selection. The critical element in this cycle is the quality of the selection criterion. Due to the labour intensive aspects of library screening, most successful molecular evolution exercises to date rely on simple, rigorous criteria to separate successful from unsuccessful candidates. Consequently the potential of molecular evolution is restricted by the need to design a simple, rigorous basis for selection.

Furthermore, in these methods, mutant candidates that present only a slight improvement in the desired property can be eliminated regardless of the possibility that such a mutant could, when combined later with another slightly or strongly improved mutant, deliver a significant improvement in the desired property. Both of these limitations of the art can be addressed by any advance that simplifies the task of library screening.

Any advance that simplifies the task involved in the library screening step has the effect of increasing the ambit of molecular evolution applications to encompass selection protocols based on subtle, less rigorous screening criteria and also can retain more slightly improved mutant candidates.

There thus exists a great need in the art for improved methods of in vitro evolution for the selection of molecules with improved biological activity, allowing the selection of molecules possessing either catalytic function or binding affinity. Suitable methods should allow the high throughput screening of a large number of molecules containing different mutations, with the selection process allowing the easy identification of molecules with improved function and the subsequent separation of the encoding genetic element.

### SUMMARY OF THE INVENTION

This invention embraces a wide variety of possible mechanisms by which compounds with a desired activity may be selected. A unifying feature of all these mechanisms is that the coding region being evolved is in the same genetic element or on the same DNA molecule as a target site for a DNA-modifying protein. Accordingly, the activity (or inactivity) of the DNA-modifying protein can be tested by evaluating the sequence of its nucleic acid substrate. In this manner, a number of different types of compounds may be selected, including improved DNA-modifying proteins, improved substrates for DNA-modifying proteins, improved ligand-receptor interactions, improved co-factor and regulatory protein activities, improved DNA-binding proteins, and so on. The methods of the invention will be referred to herein as Substrate Linked Directed Evolution (SLiDE).

According to a first aspect of the invention, there is provided a method of selecting a nucleic acid encoding a DNA-modifying protein with a desired activity against a nucleic acid substrate comprising the steps of:

a) providing a library of genetic elements in which each genetic element includes:

i) a nucleic acid sequence encoding a DNA-modifying protein, and

ii) said nucleic acid substrate;

b) incubating said library under conditions suitable for the expression and activity of its DNA modifying proteins; and

c) selecting a nucleic acid that encodes a DNA-modifying protein with the desired activity by identifying a genetic element in which the nucleic acid substrate either has, or has not been modified.

In a preferred embodiment of the invention, a nucleic acid is selected whose sequence either has, or has not been modified.

The method of this aspect of the present invention is therefore suitable for the evolution of DNA-modifying proteins with new or improved functions.

The system is set up so that a DNA-modifying protein possessing a desired phenotype causes a change in the genetic element in which it was encoded. This makes it possible to enrich for this genetic element in a subsequent step by selecting for altered nucleic acid substrate. Desirable genes are thus selectively enriched. The method can be repeated in iterative steps of mutation and selection, so that the desirable molecules are enriched in each selection step of the cycle. Genetic elements that encode molecules of interest are selected to parent the next generation.

This invention thus relies on the use of a library of genetic elements in which each genetic element encodes both a DNA-modifying protein and a substrate for that DNA modifying protein. The substrate is thus only altered in the event that the genetic element encodes an active DNA-modifying protein that recognises that particular substrate. Because the nucleic acid substrate for the DNA-modifying protein resides in or on the genetic element itself, when the substrate is altered, selection for the altered nucleic acid substrate allows the concomitant isolation of the coding information for an active DNA-modifying protein of interest.

To ensure the linkage between the encoded genetic information and the resulting phenotype that is selected, some form of compartmentalisation is required. Any method of compartmentalisation that ensures that genetic information may not be exchanged between compartments is suitable for use in the present invention.

The term “genetic element” as used herein is therefore meant to include any entity that contains or encodes genetic information and which allows the linkage of its encoded genetic information with a substrate for a DNA-modifying protein. This linkage is necessary so that it can be certain that when a genetic element is selected on the basis of a nucleic acid substrate within it having been altered (or, of course, having remained unaltered), the altered or unaltered status of that nucleic acid substrate is the definite result of the activity of the DNA-modifying protein within that same genetic element (compartment). Identification of those genetic elements in which substrate nucleic acid has been converted to product nucleic acid concomitantly identifies the genetic information that encoded an active, or activated DNA modifying protein. Of course, the reverse is also true when selecting for inactive, or inactivated DNA-modifying proteins. In the methods of the present invention, there is no covalent linkage formed between the DNA modifying protein and the nucleic acid substrate.

As used herein, the term “genetic element” may therefore be an organism such as a prokaryotic or eukaryotic cell, a bacteriophage or a virus. One in vitro system recently published in International patent application WO99/02671 reports the use of microcapsules created using water-in-oil emulsions to compartmentalise and thus isolate the components of a translation system. Such microcapsules may represent genetic elements according to the invention.

The constituent components of a reaction of interest must all be provided to each genetic element in some way to allow the reaction to take place. The only essential aspect of the method is that the nucleic acid molecule that encodes the protein whose properties are being selected for is contained within the same genetic element as the nucleic acid substrate for the DNA-modifying protein; the other components may be added exogenously if desired. The skilled reader will appreciate that there are number of potential ways in which the constituent components may be introduced into a system so that all constituents are present. For example, in the case of the genetic element entity being provided by a particular cellular organism, some or all of the components of the reaction may be expressed from the genome of the organism. In an alternative embodiment, some or all of the constituent components of the reaction may be expressed from an extrachromosomal element such as a plasmid, episome, artificial chromosome or the like. These possible arrangements may, of course, be mixed so that some of the components are expressed from the genome of the organism and some are expressed from an extrachromosomal element.

In cases where the DNA-modifying protein of interest requires the presence of other proteins for full activity, these proteins should also be included in the reaction and may be encoded by the chromosome of the cell, or in a plasmid. The proteins may be coded for by the same genetic element that encodes the DNA-modifying protein of interest, for example, on the same plasmid.

Although the substrate for the DNA-modifying protein and the nucleic acid encoding the DNA-modifying protein should be encoded in or on the same genetic element, these entities need not be encoded by the same nucleic acid molecule. For example, in the case of a library of bacterial cells, the DNA-modifying protein may be encoded on a plasmid present in each cell, whilst the substrate may be situated on the bacterial chromosome. Alternatively, the substrate may be situated on a plasmid and the DNA-modifying protein may be encoded anywhere else within the same cell, such as in the genome. In both cases, the gene that is the subject of the molecular evolution exercise, is sited next to the substrate. Because the bacterium effectively confines the components of a particular system within it and excludes proteins encoded in other cells of the library, the connection between the tested phenotype and the causative genotype is retained.

A library of genetic elements may comprise a plurality of transformed cells, each cell of which expresses a different DNA-modifying protein. The different “genotypes” may result from differences in the genomes of the organisms of the library. More usually, however, it will be more convenient to create a library of cells by transforming a preparation of cells with a library of vectors, such as a plasmid, episome, bacteriophage or viral vector library, or an artificial chromosome library. Under the appropriate conditions, transformation with plasmids, episomes or bacteriophage may be performed so as to ensure that only one type of genetic element is expressed in each cell of the library.

A library of cells should be created so that on average, only one nucleic acid type is transformed into each cell. This confines all the proteins that are expressed from that nucleic acid within the same cell and facilitates the selection of nucleic acids encoding molecules of interest; were each cell to include multiple nucleic acid molecules, then upon isolation of the cell it would not be clear which nucleic acid molecule had encoded the protein that caused the desired effect. According to the invention, any alteration of substrate nucleic acid as a result of the presence of active DNA-modifying protein will therefore be the direct result of the activity of the protein in that same cell. Selection for altered nucleic acid substrate thus selects for those cells that encode active or activated DNA-modifying protein.

Bacteriophage are also suitable as genetic elements for use in the methods of the present invention, since the step of bacterial infection may be designed under appropriate conditions such that only one bacteriophage type is sustained in each bacterial cell. This means that if the nucleic acid substrate is altered within the bacteriophage, this must be the result of the presence of active, or activated DNA-modifying protein.

To facilitate the selection of a DNA-modifying protein with the desired function, it is desirable to select from a library containing a diverse variety of genetic elements, each encoding a different DNA-modifying protein. This increases the chance that the library will contain at least one molecule with the desired characteristics.

Methods for the creation of libraries are well known in the art. For example, a cDNA library may be isolated from any organism or cell type by reverse transcription of the mRNA present in the organism or cell. A huge variety of cDNA libraries are also now available commercially. Libraries can be cloned into suitable plasmid, phage or viral vectors using standard methods in the art (see, for example, Sambrook J., Fritsch E. F. & Maniatis T. (1989) Molecular cloning: a laboratory manual. New York: Cold Spring Harbor Laboratory Press; Fernandez J. M. & Hoeffler J. P., eds. (1998) Gene expression systems. Academic Press).

In an alternative embodiment, rather than encoding a diverse number of different compounds, a library may contain a number of variants of a single type of protein. For example, if it is desired to improve or alter the properties of a particular DNA modifying protein, a library may be generated by mutagenesis of the gene encoding this protein, or by rational mutagenesis of the relevant part of the gene encoding this protein.

The term “DNA-modifying protein” as used herein is meant to include any protein whose activity causes a change in the sequence or structure of nucleic acid, so causing a change in the sequence or structure of a DNA molecule that can be used to differentiate molecules that have been altered from those that have not. In this way, the activity of a DNA-modifying protein can be assessed.

The DNA-modifying protein may be solely responsible for the alteration of substrate nucleic acid. In this, simplest, embodiment of the method, no other proteins participate in the substrate conversion process.

However, as the skilled reader will appreciate, the DNA-modifying protein may form part of a multi-protein complex that is inactive in the absence of the DNA-modifying protein of interest. For example, some DNA-modifying proteins are in fact holoproteins, made up of individual constituent proteins. In this embodiment of the invention, the complex will only be activated when all of the individual constituent proteins of the holoprotein are present in the same cell.

Examples of DNA-modifying proteins suitable for evolution using the method of the present invention include site-specific recombinases (SSRs), proteins involved in homologous recombination (HR), exonucleases, DNA methylases, DNA ligases, restriction endonucleases, topoisomerases, transposases and resolvases. All these molecules cause changes in the structure of a DNA molecule that can be followed using the techniques of biochemistry or molecular biology. Suitable examples of each protein type will be clear to those of skill in the art.

For example, this aspect of the method of the invention can be applied to any protein that is involved in the process of homologous recombination (HR). HR involves DNA rearrangement between two identical or nearly identical sequences, initiated by specific HR proteins. These proteins form a recombinase complex that when assembled is active to alter the DNA structure. Examples of suitable proteins include RecA, RecE, RecT, Redα, Redβ, eukaryotic Rad51, eukaryotic Rad52, T4 phage UvsX, T7 phage gene 6, T7 phage gene 25, Saccharomyces cerevisiae Sep1, Saccharomyces cerevisiae Dpa1, and HSV ICP8. Other suitable examples will be clear to those of skill in the art. The presence of an HR protein of the desired function can be selected by isolating genetic elements that have been rearranged by the HR event.

Restriction endonucleases may also be used in the method of this aspect of the invention. These proteins bind as homodimers to specific sites on DNA molecules. Selection of cells whose nucleic acid has been restricted at the consensus recognition site of such an enzyme allows the selection of cells that encode restriction endonucleases possessing the properties of interest. These cells can thus be discriminated from those that do not encode active restriction endonucleases.

DNA methylases may also be used in the method of this aspect of the invention. In this embodiment, the DNA methylase is either itself the ‘gene-of-interest’ (i.e. its encoding gene is mutated to create a library which can then be screened for DNA methylases of interest), or the DNA methylase may report the activity of a heterologous protein whose gene is mutated to create the library. In this latter example, this extra protein regulates the DNA methylase. The DNA methylase either methylates, or not, a substrate site on the nucleic acid near the gene of interest. The library is retrieved and cleaved in vitro with a restriction enzyme that also recognises the substrate site when it is methylated, or not methylated, as appropriate to the scheme. By using PCR primers placed either side of (a) the mutated gene and (b) the methylase substrate site; only those molecules that were not cut by the restriction enzyme will be amplified. These molecules will include successful candidate nucleic acids. These can then be used to clone into the new library for a subsequent round of screening and selection.

Preferably, the DNA-modifying protein is a protein involved in recombination, such as a SSR or HR protein, more preferably, an SSR protein. SSRs are enzymes that recognise and bind to specific DNA sequences termed recombinase targets (RTs) and mediate recombination between two RTs. This causes a change in the sequence of DNA that allows discrimination of recombined targets from those that have not been recombined.

The term “SSR” thus refers to any protein component of any recombinant system that mediates DNA rearrangements in a specific DNA locus, including SSRs of the integrase or resolvase/invertase classes (Abremski, K. E. and Hoess, R. H. (1992) Protein Engineering 5, 87-91; Khan, et al., (1991) Nucleic acids Res. 19, 851-860; Nunes-Duby et al., (1998) Nucleic Acids Res 26 391-406; Thorpe and Smith, (1998) P.N.A.S USA 95 5505-10) and site-specific recombination mediated by intron-encoded endonucleases (Perrin et al., (1993) EMBO J. 12, 2939-2947).

Preferred SSR proteins are selected from the group consisting of: FLP recombinase, Cre recombinase, R recombinase from Zygosaccharomyces rouxii plasmid pSR1, A recombinase from the Kluyveromyces drosophilarium plasmid pKD1, a recombinase from the Kluyveromyces waltii plasmid pKW1, TnpI from the Bacillus transposon Tn4430, any component of the λ Int recombination system or any other member of the tyrosine recombinases; phiC31, or any other member of the large serine recombinases; any component of Gin or Hin recombination systems, resolvase, or any other member of the serine recombinases; Rag 1, Rag 2 or any other component of the VDJ recombination system, or variants thereof, phiC31, any component of the Gin recombination system, or variants thereof. The term “variant” in this context refers to proteins which are derived from the above proteins by deletion, substitution and/or addition of amino acids and which retain some or all of the function inherent in the protein from which they are derived. Specifically, the variant could retain the ability to act as a recombinase, or it could retain protein/protein or protein/DNA interactions critical to the recombination reaction, or to the regulation of the recombination reaction.

The recombinase protein may not itself be active as a recombinase enzyme, but may form a component of a recombinase complex, such as, for example, a component of the λ Int or Gin recombination systems. In this embodiment of the invention, the remaining components of the recombinase complex should be present in the cell so that when the recombinase, component is expressed, the recombination event is able to take place.

The property being selected for may be an improved catalytic efficiency, or an increased rate of substrate turnover. Selection might therefore be under conditions of increased stringency, for example, using shorter incubation times, such that only the most efficient DNA modifying proteins would alter the nucleic acid substrate in the time period allowed.

In another alternative, the method may be used to select for novel DNA-modifying proteins that recognise a specific nucleotide consensus sequence. This would involve the screening of cells transformed with a library of candidate cells transformed with a library encoding DNA-modifying proteins. Selection would be by including a nucleic acid substrate of the required sequence within each member of the library and isolating those cells in which the nucleic acid substrate, and more specifically, the sequence of the nucleic acid substrate, had (or had not) been altered. In this eventuality, each member of the library should contain as RTs, two portions of nucleic acid of the appropriate sequence that a novel DNA modifying protein should bind to. The presence of an SSR protein that is capable of causing rearrangement between these sequences can be tested by selecting those cells in which recombination has taken place.

In a further example, the method may be used to select for novel restriction enzymes that recognise a specific nucleotide sequence. This would involve, for example, the construction of a genetic element such as a plasmid that contains a library of genes encoding candidate restriction enzymes together with a gene that encodes for antibiotic resistance. In one embodiment of this example, the coding region for the antibiotic resistance gene may be disrupted so that it does not express antibiotic resistance. The candidate restriction enzyme site may be placed at the site of breakage. Either side of the breakage site, a section, for example, at least 6 base pairs, of the coding region of the antibiotic resistance gene may be repeated. If the candidate restriction enzyme cleaves the site, the antibiotic resistance gene will be reconstituted by double strand break repair through the repeated section, meaning that cells exhibiting this phenotype may be selected by resistance to antibiotic. This particular example requires that the host cell be competent for double strand break repair. Such a function can be provided in Escherichia coli by RecE/RecT, Recα/Recβ or RecA.

Other desirable properties for selection will be clear to the skilled reader.

In order to improve the chances of successfully selecting for the desired DNA-modifying protein activity, in the selection step of the method, the library should be incubated under conditions that are suitable for the activity of the DNA-modifying protein. Accordingly, there should be present in the system the appropriate transcriptional and translational machinery to allow expression of these proteins from their encoding genes. This machinery will in most cases be derived from the cells of the library.

Conditions should also be used that allow for expression of the DNA modifying proteins and that are optimal for its activity. Such conditions will include appropriate temperature, the inclusion of necessary concentrations of co-factors, solution ions and so on. Suitable conditions will be clear to those of skill in the art.

The design of a suitable nucleic acid substrate for the DNA-modifying protein will depend on the particular DNA-modifying protein being used. For example, in the case of a SSR enzyme, the substrate will include two recombinase targets (RTs) whose constituent sequences are recognised by the SSR enzyme. The presence of active SSR protein in the cell will cause rearrangement of the genetic element between the RTs, so giving a product that can be differentiated from substrate.

Once altered by active DNA modifying protein, the nucleic acid substrate must differ in some respect to allow its discrimination from unaltered substrate. In this manner, cells in which a successful reaction has taken place (which thus encode a candidate compound with the desired properties) can be identified. Suitable methods for the selection of altered nucleic acid template will be clear to those of skill in the art and will, of course, depend on the property of the DNA-modifying protein that is being utilised. Any method that allows the identification of altered DNA sequence or structure will thus be appropriate. Examples include restriction analysis, single-stranded conformational polymorphism (SSCP) analysis, restriction fragment length polymorphism analysis (RFLP), PCR-based methods and SDS-PAGE. As the skilled reader will be aware, the highly accurate techniques of SSCP and PCR allow the differentiation of nucleic acid molecules that vary by only one nucleotide. Accordingly, the nucleic acid product may differ from nucleic acid substrate by only one nucleotide substitution, deletion, or insertion. As the skilled reader will be aware, restriction analysis and susceptibility to certain chemicals can be used to distinguish the presence or absence of covalent chemical modifications, such as methylation, at a single nucleotide, or more.

What is common to all the methods that are the subject of the present invention is that no covalent link is formed between the DNA modifying protein and the nucleic acid substrate. Selection of altered (or unaltered) nucleic acid substrate in all, cases relies on changes in the sequence or structure of the nucleic acid itself (preferably sequence) and not on isolating a compound that is bound covalently to the nucleic acid substrate.

With respect to methods that utilise recombinases as DNA-modifying proteins, methods for determining recombinase activity include the detection, either direct or indirect, of recombination or changes in the recombination rate between DNA target sites. Direct measurements of the physical arrangement of the target sites may utilise techniques such as gel electrophoresis of DNA molecules, Southern blotting or PCR-based methods. Indirect measurements may be by assessing the properties encoded by regions of DNA that carry recombinase target sites before or after recombination. For example, recombination could excise a cytotoxic gene from the genetic element encoding the recombinase and thus recombination could be measured in terms of resistance of a host cell to a toxin.

In most instances, the more convenient and adaptable techniques for examination of modified or unmodified nucleic acid sequences will be those based on the polymerase chain reaction (PCR). This technique allows the specific amplification of altered DNA templates using primers that either only bind to altered DNA template and not to unaltered DNA template or, after binding can only generate a PCR product on the altered but not unaltered DNA template. In the latter case, a further processing step before PCR, such as restriction enzyme cleavage, may be useful. The amplified template can then be purified and the successful candidate genes cloned back into a suitable genetic element that can be used to parent the next generation in the selection process.

In many instances, selection of nucleic acid sequences encoding successful candidates can be based on changes in gene expression caused by the change in the substrate due to the activity of the DNA modifying protein. For example, with appropriate design of the substrate, the change imposed by the DNA modifying protein could activate the expression of an antibiotic resistance gene, allowing selection with antibiotics for the successful candidate, or activate the expression of a phenotypic marker gene, such as a gene encoding green fluorescent protein or b-galactosidase, permitting a physical enrichment method such as FACS (fluorescent activated cell sorting). Since any molecular evolution exercise is a search for a rare event, or more often, for a combination of rare events, in a vast background of other possibilities, any improvement that can be made to screen through vast numbers of candidates to identify a successful event will be useful. Hence, the combination of more than one of the above screening procedures, for example, a FACS step followed by a PCR step, will facilitate the identification of advantageous candidates that can then serve to parent the next round.

Selection may either be for altered nucleic acid substrate, or unaltered nucleic acid substrate.

As with all in vitro evolution methods described to date, in order to optimise the property of the DNA-modifying protein which is being selected for, more than one selection step is generally necessary. Consequently, the candidates chosen on the basis of successful (or unsuccessful) modification of nucleic acid substrate are selected to parent a next generation of candidates and the process is repeated.

The improved selection techniques that form part of the invention permit the simple use of reiterative molecular evolution cycles so that large pools of potential candidates can be carried through a series of repetitions. In the first cycle, such a pool will be predominantly contaminated with unsuccessful candidates. However, upon reiterative cycling, the content of the pool will increasingly become populated by successful (“fitter”) mutant candidates. Hence, by simplifying the labour intensive task of library screening so that it can be readily and reiteratively applied, the method of the invention allows non-rigorous selection criteria to be used, so that mutations that deliver subtle improvements can be retained. After a series of reiterative cycles, the pool of successful candidates can be taken to create a new library that is used to start a new series of reiterative cycling under a more stringent selection criterion.

In order that the selected molecules “evolve” between selection steps, the selected candidates may be mutagenised so as to introduce mutations into the sequence and create a new library of candidates for testing in the next round of selection. For example, it may be preferable to start with one particular DNA modifying protein sequence that encodes a protein with properties that are similar to those that are desired. By mutating the sequence of this protein type to create a library of variant proteins, a biased library is obtained that provides a useful point from which to start the selection process. The selection process may then be performed in a number of iterative cycles; by increasing the stringency of selection at each round, the gene pool will gradually be enriched for proteins that possess the desired properties.

Suitable methods of mutagenesis will be known to those of skill in the art and include point mutagenesis (error-prone PCR, chemical mutagenesis, the use of specific mutator host strains), recursive ensembel mutagenesis (Delagrave and Youvan (1993) Bio-Technology, 11: 1548-1552), combinatorial cassette mutagenesis (Black et al., 1996), DNA shuffling (Stemmer et al., 1994) or by codon substitution mutagenesis. For a review of recent improvements in processes for in vitro recombination, see Giver and Arnold, 1998 (Current opionion in chemical biology, 2(3): 335-338).

It may be preferable to direct the mutagenesis of candidates, for example, to target mutations to a particular area or domian of a molecule that is being selected. This can most suitably be done using oligonucleotide-directed mutagenesis or by PCR using, for example, degenerate oligonucleotides that bind to a specific nucleotide sequence in the nucleic acid coding region.

Preferably, at least two cycles of mutagenesis and selection are performed, although the possibility of automation may allow the use of 1000 or more cycles, if necessary.

According to a still further embodiment of this aspect of the invention, there is provided a nucleic acid molecule encoding a DNA modifying protein identified according to any of the embodiments of the invention described above. The invention also provides a DNA modifying protein encoded by such a nucleic acid molecule. Examples of types of DNA modifying proteins that may be selected using these methods include site-specific recombinases, enzymes involved in homologous recombation, exonucleases, DNA methylases, DNA ligases, restriction endonucleases, topoisomerases, transposases and resolvases. Particular examples include the mutant Cre and Fre recombinases described in the examples contained herein, in particular, Fre 3, 5 and 20.

In a second aspect of the invention, molecules that regulate, modulate, interfere with or enhance (hereafter encompassed by the terms “regulate”, “regulated” and “regulation”) the activity of a DNA modifying protein can be selected using the method of substrate linked directed evolution described above. In all cases, a DNA modifying protein acts upon a site that is physically linked to the coding region of the molecule that is selected in the directed evolution exercise. The action of the DNA modifying protein on the specific DNA sequence reflects the activity of the molecule that regulates the DNA modifying protein. Successful candidate molecules are identified by the alteration, or lack of alteration, in the substrate that is physically linked to the nucleotide sequence that encodes the successful candidate. In this second aspect of the invention, it should be noted that the nucleic acid sequences that encode the DNA modifying protein need not be physically linked to the substrate and nucleic acid sequences encoding the molecule that is selected. One exception is the case in which the coding region of the DNA modifying protein is fused to the coding region of the molecule that is being selected to produce a fusion molecule between the two.

According to this aspect of the invention, there is provided a method of selecting one or more genetic elements encoding a candidate molecule having a desired activity, or having the ability to direct the synthesis of a candidate molecule having a desired activity, said method comprising the steps of:

a) providing a library of genetic elements, in which each genetic element includes:

i) a nucleic acid sequence encoding a candidate molecule for possession of the desired biological activity, or having the ability to direct the synthesis of a candidate molecule having a desired activity; and

ii) a nucleic acid sequence which constitutes a substrate for a DNA-modifying protein;

iii) a protein with DNA-modifying activity;

wherein the activity of said DNA-modifying protein is regulated by the activity of said candidate molecule, such that modification of the nucleic acid substrate only occurs in the event that the nucleic acid sequence encodes or directs the synthesis of a candidate molecule having the desired activity;

b) incubating said library and said protein with DNA-modifying activity under conditions that are suitable for its DNA-modifying activity; and

c) selecting a nucleic acid that encodes a candidate molecule with the desired activity by identifying a genetic element in which the nucleic acid substrate either has, or has not been modified.

This system is arranged so that a molecule possessing a desired activity effects a change in the particular genetic element in which it was encoded. Preferably, the change is effected in the sequence of the genetic element. This makes it possible to enrich for the nucleic acid encoding this molecule in a subsequent step by selecting for genetic elements in which the change has taken place. Desirable genes are thus selectively enriched. As with many methods of in vitro evolution, the method can be repeated in iterative steps of mutation and selection, so that the desirable molecules are enriched in each selection step of the cycle. At each step, genetic elements that encode molecules of interest are selected to parent the next generation.

This invention relies on the use of a genetic element that includes both a nucleic acid sequence encoding a molecule that is a candidate for possessing the desired activity, or that participates in a metabolic pathway that produces a molecule with desired activity, and a nucleic acid sequence that constitutes a substrate for a DNA-modifying protein. The candidate molecule and nucleic acid substrate are confined within the same system. The system is designed such that a successful interaction between the candidate molecule and its target is reflected by the alteration of the activity of a protein that possesses DNA-modifying activity. The nucleic acid substrate is thus only altered in the event that the system contains an activated DNA-modifying protein that recognises the nucleic acid substrate. This enables the identification of genetic elements that include a nucleic acid encoding a molecule with the desired properties; selection of these genetic elements allows the concomitant isolation of the coding information for the molecule of interest.

For example, selection of altered nucleic acid substrate allows the isolation of the coding information for a DNA-modifying protein that has been activated by some molecular event. Selection of unaltered substrate selects for inactive DNA-modifying protein and thus is useful for isolating inhibitors of DNA-modifying proteins, or DNA binding proteins that occlude the DNA-modifying protein from binding to and altering its substrate.

The occurrence of a successful molecular interaction between candidate molecule and its target may be assessed by incubating the genetic element under conditions suitable for the expression and activity of each component necessary for the interaction and then analysing that genetic element for the presence, or absence, of an altered nucleic acid substrate. Identification of those genetic elements in which the desired reaction has taken place allows the isolation of the genetic information that encoded a molecule that participates successfully in the interaction.

In one embodiment of this example, the DNA modifying protein is expressed in a form which either is incapable of acting upon the substrate because it is inhibited by a specific molecular mechanism, or acts upon the substrate unless it is inhibited by a specific molecular mechanism.

The specific molecular mechanism can be directed towards the DNA modifying protein itself, its activity as a protein or any component that is required for its activity as an protein. Alternatively, the specific molecular mechanism can be directed towards the substrate of the DNA modifying protein.

Nucleic acid sequences that encode candidate molecules that relieve or impose the inhibition, or nucleic acid sequences that encode molecules that participate in the synthesis of cofactors, including lipids, sugars, steroids, peptides and any other product of a metabolic pathway that relieves or imposes the inhibition, can be identified from libraries of candidate molecules placed next to the substrate.

In another embodiment of this aspect of the invention, the DNA modifying protein is expressed in a form which either does not act upon the substrate without a cofactor or acts upon the substrate unless a cofactor interferes with it. Nucleic acid sequences that encode part or all of candidate cofactors, or encode molecules that participate in the synthesis of cofactors, including lipids, sugars, steroids, peptides and any other product of a metabolic pathway that serves as part or all of the cofactor, can be identified from libraries of candidates using this method.

In this aspect of the invention, the DNA-modifying protein may be encoded in the same genetic element as the nucleic acid substrate and the nucleic acid that encodes the candidate molecule. The DNA-modifying protein may therefore be encoded, for example, in the genome of a cell, or it may be encoded by an extrachromosomal element. In the latter case, the DNA-modifying protein may be encoded on the same extrachromosomal element as the nucleic acid substrate and/or the nucleic acid that encodes the candidate molecule. As the skilled reader will be aware, provided that the three components of the DNA-modifying reaction are confined within the same compartment, to the exclusion of reaction components encoded in other genetic elements, the required link between genotype and phenotype will be retained.

In this aspect of the invention, the activity of the DNA-modifying protein should be linked to the activity of the candidate molecule of interest. By this is meant that the candidate molecule must in some way affect the activity of the DNA-modifying protein, such that the activity of the DNA-modifying protein is either raised or lowered specifically as a result of a desired property of the candidate molecule. In this manner, if the candidate molecule possesses a desired activity, the particular cell that encoded that same candidate molecule may be isolated on the basis of the sequence of the nucleic acid substrate for the DNA-modifying protein.

There are a large number of ways by which the activity of a candidate molecule may be linked with the activity of a DNA-modifying protein, as the skilled reader will appreciate. For example, the DNA-modifying protein may be inactive in the absence of a candidate molecule of the desired activity. The molecule may bind directly or indirectly to the DNA-modifying protein and thereby affect its activity. An example of such an interaction might be the interaction of a co-factor with a DNA-modifying protein or the interaction of any other protein type whose activity is essential for the proper functioning of the DNA-modifying protein.

The candidate molecule may interact with the DNA-modifying protein through an intermediary effector molecule. For example, the DNA-modifying protein may be associated with a regulatory domain that represses the activity of the DNA-modifying protein in the absence of a cognate ligand. In this aspect of the invention, the candidate molecule being selected for might therefore be a ligand with a novel or improved affinity for the regulatory domain. In this respect, the discussion below of the use of fusion proteins, particularly those with the properties disclosed in European patent 0 707 599, is particularly relevant. Selection may either be for altered nucleic acid substrate, or unaltered nucleic acid substrate. For example, in the case of selecting for an inhibitor molecular that possesses inhibitory activity against a DNA-modifying protein, selection of the most effective inhibitors will involve selecting for those cells in which the DNA-modifying protein has been inactive, and thus in which the nucleic acid substrate remains unaltered. However, in most circumstances, selection will be for cells whose nucleic acid substrates have been altered.

According to a still further embodiment of this aspect of the invention, there is provided a nucleic acid encoding a candidate molecule selected according to any one of the methods of the invention described above. The invention also provides a candidate molecule encoded by such a nucleic acid molecule. In particular, such molecules include small drug molecules, ligands, receptors, DNA binding proteins, inhibitors, cofactors and activators of DNA modifying proteins.

In a third aspect of the invention, ligand or receptor molecules with novel, or altered properties can be selected.

In a preferred embodiment of this aspect, there is provided a method of selecting for a nucleic acid encoding a receptor molecule with affinity for a target ligand, comprising the steps of:

a) providing a library of genetic elements in which each genetic element includes:

i) a nucleic acid sequence which constitutes a substrate for a DNA modifying protein;

ii) a nucleic acid sequence encoding a fusion protein comprising a DNA modifying protein fused to a candidate receptor molecule, wherein the DNA modifying activity of the protein is low or high in the absence of ligand binding to said receptor molecule and is induced, repressed or altered by binding of ligand to receptor;

b) incubating said library under conditions suitable for the activity of its DNA modifying proteins;

c) exposing said library to ligand, or to a mixture of different ligands;

d) selecting a nucleic acid that encodes a receptor with the desired ligand binding activity by identifying a genetic element in which the nucleic acid substrate either has, or has not been modified.

In another preferred embodiment of this aspect, there is provided a method of selecting for a nucleic acid molecule encoding a ligand with affinity for a target receptor comprising the steps of:

a) providing a library of genetic elements, in which each genetic element includes:

i) a nucleic acid sequence which constitutes a substrate for a DNA modifying protein;

ii) a nucleic acid sequence which encodes a candidate ligand;

b) incubating said library under conditions suitable for the activity of its DNA modifying proteins; and

c) exposing said library to a fusion protein comprising a DNA modifying protein fused to the target receptor, wherein the DNA modifying activity of the protein is low or high in the absence of ligand binding to said receptor and is induced, repressed or altered by binding of ligand to receptor;

d) selecting a nucleic acid that encodes a ligand with the desired activity by identifying a genetic element in which the nucleic acid substrate either has, or has not been modified.

In both these aspects of the invention, a nucleic acid is preferably selected whose sequence either has, or has not been modified.

The fusion protein comprising DNA modifying protein and target receptor may be encoded by the genetic element of part a).

These embodiments of the invention thus provide for the selection of either component of a desired binding interaction. As for the first aspect of the invention set out above, a library of cells is used, each of which includes a nucleic acid substrate for a DNA-modifying protein. However, in this embodiment of the invention, each cell encodes a fusion protein that comprises a DNA modifying protein, fused to part or all of a receptor molecule that exhibits affinity for a ligand. The fusion protein is designed such that the activity of the DNA modifying protein is inhibited in the absence of ligand binding to the receptor and is induced or altered by the binding of ligand to receptor, or is active in the absence of ligand binding to the receptor and is inhibited or altered by binding of ligand to receptor. Expressed ligands bind to and activate or inhibit the DNA modifying protein only if the ligand shows high affinity for its target receptor. Consequently, only the occurrence of a successful binding interaction between ligand and receptor results in the alteration of the substrate nucleic acid in the genetic element. In the absence of a ligand of the required binding affinity, the substrate remains unchanged, or alternatively is changed, depending on whether the ligand represses or induces the activity of the DNA modifying protein.

Cells in which a productive reaction does not take place will thus not be selected for further rounds of selection.

Preferably, the activity of the DNA-modifying protein part of the fusion protein is altered by the binding of ligand to the receptor domain by a factor of at least 10, more preferably of at least 20 and most preferably of at least 40.

As with the method of the first aspect of the invention, to ensure that the ligand giving a productive reaction is encoded by the same cell in which the modification of nucleic acid substrate took place, the reaction must take place in an enclosed (compartmentalised) system. This ensures that the fidelity of the link between phenotype and genotype is conserved. Again, it should be reiterated that according to the methods of the present invention, there is no covalent linkage formed between the DNA modifying protein and the nucleic acid substrate.

By the term “ligand” is meant any peptide or polypeptide ligand that exhibits affinity for a target receptor. This term is meant to include peptides that form an epitope with binding affinity for a target. Examples of suitable epitopes will be clear to the skilled reader and, in particular, will include molecules with binding affinity for antibodies, for receptors, for bioligands (for example, biotin and avidin), for distinct protein domains (for example, an SH3 domain), for other peptide epitopes, for consensus sequences in protein molecules (for example, a kinase recognition site), or for a specific cell type (for example, a lymphocyte). Other examples will be clear to those of skill in the art.

Polypeptide ligands include any polypeptide that interacts specifically with another protein and include, for example, receptor domains, antibody domains, DNA binding protein domains, effector domains, protease domains and transcription factors.

The term “ligand” as used herein is also intended to include any synthetic molecule, or product of a biosynthetic pathway, that can serve as a ligand. In the case of a synthetic molecule, this must be added in an effective concentration and at a stage in the method described, so as to influence the activity of the DNA modifying protein before the DNA modifying protein can act on its substrate. In the case of a ligand that is the product of a biosynthetic pathway, the biosynthetic pathway must be operational in the compartment in which the DNA modifying protein is present, before the ligand activity is manifested.

The term “receptor” is meant to include any molecule, preferably a polypeptide molecule, that possesses the ability to bind to a ligand as this term is defined above. This term therefore includes all or part of an antibody, a membrane receptor, a nuclear receptor (for example, a hormone receptor), an enzyme, a DNA binding protein, a protein domain (for example, an SH3 domain), a transcription factor and so on.

A number of different types of DNA modifying protein may be used in this aspect of the invention, as discussed above for the first aspect of the invention. The method of this aspect of the invention is particularly well suited for use with DNA modifying proteins that are involved in recombination, particularly site-specific recombinases. In a preferred embodiment, successful binding of ligand to the receptor portion of the fusion protein, the recombinase protein is activated, binds to its recognition sequences present in the DNA of a cell (the substrate) and mediates recombination between these sequences. This causes a change in the DNA sequence in the cell that allows recombined templates to be discriminated from unrecombined templates.

In a preferred embodiment, the fusion protein may be designed such that its DNA modifying activity is inhibited in the absence of ligand binding to receptor and is induced or altered by the binding of ligand to receptor. Expressed ligands bind to and activate the DNA modifying protein only if the ligand shows high affinity for its target receptor. Consequently, the occurrence of a successful binding interaction between ligand and receptor results in the alteration of substrate nucleic acid by the activated DNA-modifying protein.

In a preferred embodiment, fusion proteins should comprise an amino acid sequence of a DNA-modifying protein or an active fragment thereof, physically attached to the amino acid sequence of a ligand binding domain (LBD) of a receptor. By “active fragment” is meant any fragment of a DNA modifying protein that retains the ability to modify a nucleic acid substrate.

Preferably, the receptor portion of the fusion protein is a nuclear receptor, or is the LBD of a nuclear receptor, meaning any molecule, which may be glycosylated or unglycosylated, that possesses an ability to bind to ligand. Specifically, the term refers to those proteins that display functional or biochemical properties that are similar to the functional or biochemical properties displayed by receptor proteins with respect to ligand binding (Whitelaw et al., 1993). Upon binding to ligand, nuclear receptors become active, or altered, transcription factors.

More specifically, nuclear receptors may be related by their amino acid sequence to the LBDs of steroid hormone receptors, for example, a receptor that is recognised by steroids, vitamins or related ligands. Examples of suitable nuclear receptors are listed in Laudet et al., 1992, which is hereby incorporated by reference. Preferably, the nuclear receptor is a steroid hormone receptor, more preferably, a glucocorticoid, oestrogen, progesterone, or androgen receptor. Mutant receptor derivatives that retain sufficient relatedness to nuclear receptor amino acid sequences so as to be identifiable as related using the methods described by Laudet et al are included in this term.

Preferably, the DNA-modifying protein is fused to the receptor or ligand binding domain thereof by means of genetic fusion. The fusion protein may thus be a linear genetic fusion encoded by a single nucleic acid molecule. However, fusion proteins may be linked by other means, for example, through a spacer molecule that possesses reactive groups (for example, sulphydryl groups), that are covalently bound to both the receptor domain and the DNA-modifying protein domain.

In cases of genetic fusions, the attachment of the receptor and DNA-modifying protein components may be achieved using a recombinant DNA construct that encodes the amino acid sequence of the fusion protein, with the DNA encoding the receptor domain placed in the same reading frame as the DNA encoding the DNA-modifying protein, preferably either at the amino or carboxy termini of the DNA-modifying protein. More preferably, the receptor domain is fused to the C-terminus of the DNA-modifying protein. In an especially preferred embodiment of this aspect of the invention, the receptor is fused to the DNA-modifying protein through a peptide linker that consists predominantly of hydrophilic acids and that preferably has a length of between 4 and 20 amino acids.

As the skilled reader will appreciate, it is not required that the complete receptor be present. It is sufficient that the amino acids that bind the ligand are fused to the DNA-modifying protein. For example, it is known that the LBD of a receptor can be separated from the rest of the protein and fused to a DNA modifying protein, conferring ligand regulation onto the resulting fusion proteins. For the glucocorticoid and oestrogen receptors, the domain that binds ligand has been fused to other transcription factors and also to oncoproteins, rendering the fusion proteins dependent on the relevant ligand for their activity (Webster, et al., 1988; Kumar et al., 1987; Picard et al., 1988; Eiliers et al., 1989; Superti-Furga et al., 1991; Burk and Klempenauer, 1991; Boehmelt et al., 1992).

Specific examples of suitable fusion proteins that comprise a nuclear receptor portion and an SSR portion are described in the following references, the contents of which are incorporated herein in their entirety. European patent EP-B-0 707 599; Schwenk et al., (1998) Nucleic Acids Res 26,1427-32; Kellendonk et al., (1996) Nucleic Acids Res. 24. 1404-1411; Nichols et al., (1997) Mol. Endocrinol. 11, 950-961; Nichols et al., (1998) EMBO J 17, 765-773; Logie et al., (1998) Mol. Endocrinol. 12, 1120-1132; Feil R, et al. (1996) P.N.A.S. USA, 93, 10887-90; Brocard et al (1997) P.N.A.S. USA 94: 14559-14563.

In EP-B-0 707 599, binding of ligand to the receptor portion of the fusion protein is demonstrated to allow activation of the recombinase portion of the molecule. This disclosure also demonstrates that SSR-LBD fusion proteins can coexist with target sites without recombination occurring since these proteins require ligand binding to the LBD for recombinase activity. The recombinase activity of the described SSR-LBD fusion proteins, in the absence of the relevant ligand, is at least 200× less active than wild type recombinase activity. Upon presenting the SSR-LBD fusion proteins with the relevant ligand, recombinase activity is induced to more than 20% of wild type, that is, equal to or greater than 40× induction. This means that recombination can be regulated in any experimentally-manipulatable organism by presenting the relevant ligand.

Equivalent examples to the systems described in EP-B-0 707 599 include ligand-mediated dimerisation domains (Spencer et al., (1993) Science 262 1019-24), ligand binding factors from prokaryotes, such as the tetracycline repressor (Gossen et al., (1994) Curr Opin Biotechnol 5 516-20), ligand binding domains of antibodies, membrane receptors, nuclear receptors (for example, a hormone receptor), enzymes, DNA binding proteins, specific protein domains (for example, an SH3 domain), and transcription factors may be used. Other examples of LBDs for which the cognate ligand is known will be clear to those of skill in the art.

Preferably, the LBD portion of the fusion protein is a nuclear receptor, or is the LBD of a nuclear receptor, meaning any molecule, which may be glycosylated or unglycosylated, that possesses an ability to bind to ligand. Specifically, a LBD may be any protein that displays functional or biochemical properties that are similar to the functional or biochemical properties displayed by receptor proteins with respect to ligand binding (Whitelaw et al., 1993). Upon binding to ligand, nuclear receptors become active, or altered, transcription factors.

LBDs may be related by their amino acid sequence to the LBDs of steroid hormone receptors, for example, a receptor that is recognised by steroids, vitamins or related ligands. Examples of suitable hormone receptors are listed in Gronemeyer and Laudet, (1995) Protein Profile, 2: 1173-308; Ashok et al., (1998) P.N.A.S. USA 95: 2761-6; Hahn et al., (1997) P.N.A.S. USA 94: 13743-8.

Preferably, the LBD is from a glucocorticoid, oestrogen, progesterone, mineralocorticoid, ecdysone or androgen receptor. Mutant receptor derivatives that retain sufficient relatedness to nuclear receptor amino acid sequences so as to be identifiable as related using the methods described by Laudet et al (1992) EMBO J. 11: 1003-1013 are included in the term LBD.

In a particularly preferred embodiment, Flp or Cre recombinase is fused to the LBD of the oestrogen, glucocorticoid, progesterone or androgen receptors (Gronemeyer and Laudet, (1995) Protein Profile; 2 1173-308; also Beato, 1989). Other preferred embodiments include fusing Flp recombinase, TrpI recombinase, R recombinase, or SSRs from Kluyveromyces drosophilarium or Kluyveromyces waltii to these LBDs.

Another preferred embodiment involves regulating one or more components of an SSR complex to these LBDs, in particular, components of the λ Int or Gin recombination systems. However, it is not intended that the invention be limited to known recombinases and recombination complexes and or to known nuclear receptor LBDs. Rather, the strategy of this embodiment of the invention, involving fusing recombinases, or components of recombination complexes, to LBDs or nuclear receptors is applicable to any fusion combination of these proteins which display the desired characteristics readily identifiable without undue experimentation on the part of a skilled person.

As discussed for the method of the first aspect of the invention, the term “genetic element” as used herein is meant to include any entity that contains or encodes genetic information and which allows the linkage of its encoded genetic information with a substrate for a DNA-modifying protein. Particularly suitable genetic elements include the chromosome, or one of the chromosomes, of prokaryotic or eukaryotic cells, bacteriophages or viruses, or an episome or extrachromosomal element that can be maintained in prokaryotic or eukaryotic cells, or any DNA or RNA element that can be maintained in a prokaryotic or eukaryotic cell, or a synthetic compartment. Vectors that direct extrachromosomal maintenance of DNA or RNA molecules in prokaryotes, eukaryotes or synthetic compartments are particularly suitable. In each case, an essential part of this invention is the physical linkage between a substrate site for a DNA modifying protein and the nucleic acid sequences that encode for a molecule whose properties are selected. In a preferred embodiment, in each individual cell, only one type of ligand is expressed, encoded by the DNA in the organism itself, for example, in the bacterial chromosome. Subsequent isolation of cells in which nucleic acid substrate has been altered by the DNA-modifying protein, itself activated by the ligand-receptor binding event, enables the isolation of the genetic information that encoded the active ligand or receptor.

According to a still further embodiment of these aspects of the invention, there is provided a nucleic acid molecule encoding a receptor or a ligand identified according to any of the embodiments of the invention described above. The invention also provides a receptor or a ligand encoded by such a nucleic acid molecule.

The molecular evolution approaches discussed above are cyclical processes, and aspects of each cycle are amenable to automation. In preferred embodiments, for all of the aspects of the invention that are described above, the current labour-intensive task of library screening through reiterative cycles may be automated.

Various aspects and embodiments of the present invention will now be described in more detail by way of example, with particular reference to the isolation of novel DNA binding proteins. It will be appreciated that modification of detail may be made without departing from the scope of the invention.

### BRIEF DESCRIPTION OF THE FIGURES

FIG. 1: Schematic representations of the invention.

FIG. 2: Altering the DNA sequence specificity of a site-specific recombinase.

FIG. 3: a) Nucleotide sequence of loxP and loxH sites.

b) Schematic presentation of the evolution strategy with vector pEVO10. Relevant restriction sites and primers used in PCR reactions are indicated. Grey triangles show recognition target sites for Cre recombinase (loxP). Open triangles depict loxH sites. Coding sequences for proteins and the origin of replication are shown. Expression of the recombinase in cells leads to either, recombination through the two loxP sites, recombination through two loxh sites, or to no recombination (not shown). Recombinases that have recombined the two loxH sites can be identified from the pool of recombinases by digesting isolated plasmid DNA with the restriction enzyme Ndel followed by PCR amplification with indicated primers. The amplified fragments are shuffled and cloned back into the original pEVO10 vector to start the next generation.

FIG. 4: a) Recombination of the pEVO vector series by Cre and libraries at different generation cycles. Plasmid DNA was extracted from bacteria and ran on a 0.7% agarose gel. The line with two triangles indicate the unrecombined state of the plasmid, whereas the line with one triangle depicts the plasmid after recombinase mediated recombination. M-1kb marker, 1-pEVO-loxP²-Cre grown in LB, 2=pEVO-3-Cre grown in LB, 3=pEVO-6-Cre grown in LB, 4=pEVO-3-Cre grown in 5·g/ml L-arabinose, 5-pEVO-6-Cre grown in 5·g/ml L-arabinose, 6=pEVO-3-Lib10 grown in LB, 7=pEVO-6-Lib10 grown in LB, 8=pEVO-loxP², 9-pEVO-3, 10=pEVO-6.

b) Changed recombination specificity of Fre3 illustrated utilizing a lacZ recombination reporter assay. DH5· cells harbouring the indicated reporter plasmids (pSV-paX, or pSV-paH) and pBAD33-Cre (Cre), or pBAD33-Fre3 (Fre3) grown at 50·g/ml L-arabinose. Cells were plated on X-gal containing plates. Recombination removes the promoter driving LacZ, resulting in white cells.

c) Southern blot of recombinases Cre, Fre20, Fre1, and Fre3 cloned into pEVO-10 and grown at 25 μg/ml L-arabinose. Harvested plasmid DNA was digested with BsrGI and NdeI and hybridized with a vector specific probe (see also FIG. 4B). Plasmids that have undergone recombination through the loxH sites (loxH, 5321 bp), through the loxP sites (loxP, 3390 bp) and unrecombined DNA (unrec., 4321 bp) are shown. The quantification as determined by phosphoimager analysis is depicted below the image.

FIG. 5: Recombinase mediated integration assay.

a) Schematic presentation of site specific integration of plasmid PIRate-loxH into pEVO-Fre3. Coding sequences for protein and the origin of replication are shown.

b) Colonies obtained on kanamycin plates with indicated plasmid mixtures.

c) Integration efficiencies of pIRate-loxH (white), or pIRate-loxP (black) into indicated pEVO10-recombinase vectors.

FIG. 6: Recombinases assayed in mammalian cells.

a) Plasmids expressing the depicted recombinases from the PGK promoter were co-transfected with the recombination reporter plasmids pSVpaX (loxP sites) or pSVpaH (loxH sites) into CHO cells. Illustrations of plasmids pSVpaX, pSVpaH, and pSVpaZ is presented. White triangles depict loxH sites, grey triangles loxP sites, and black triangles FRT sites. SV40═SV40 early promoter; pac=puromycin acetytransferase. Control1 shows cells transfected with the reporter plasmids pSVpaX, or pSVpaH only. Control2 shows cells transfected with the recombined form of the repoter plasmids pSVpaXΔ, or pSVpaHΔ (100% recombination).

b) Recombination efficiency of indicated recombinases and reporter plasmids in CHO cells.

FIG. 7: Sequence comparison of selected mutants. Amino acid changes found in displayed mutants are shown in bold. Secondary structure elements found in the x-ray structure are indicated as cylinders (α-helices A-N) and arrows (β-sheets 1-5). Amino acids shown to contact DNA in the crystal structure are marked with an asterix.

FIG. 8: Mapping of Fre3 mutations onto the Cre crystal structure.

FIG. 9: Altering the DNA sequence specificity of an endonuclease.

FIG. 10: Improving the efficiency of proteins that mediate DNA repair.

FIG. 11: Improving the efficiency of proteins that mediate homologous recombination.

FIG. 12: Schematic illustration of the application of the method of the invention to a gene of interest that is not a DNA modifying enzyme, rather one that influences the activity of a DNA modifying enzyme.

FIG. 13: Schematic illustration of the application of the method of the invention to the case where a gene of interest is not a DNA modifying enzyme, rather one that influences the activity of a DNA modifying enzyme when it is fused to the DNA modifying enzyme.

FIG. 14:

A. Scheme of a plasmid vector for application of a method according to the invention (SLiDE) in Saccharomyces cerevisiae.

B. DNA sequence for 22-GFP/ER251.

FIG. 15: Control experiments with 22-GFP/FLP to establish that FLP recombination induces GFP expression, which can be then be used in FACS (fluorescent activated cell sorting) as a first, phenotypic screen for the method of the invention (SLiDE).

FIG. 16: A variety of nuclear receptor LBDs were tested in yeast for repression of FLP.

### EXAMPLES

Methods

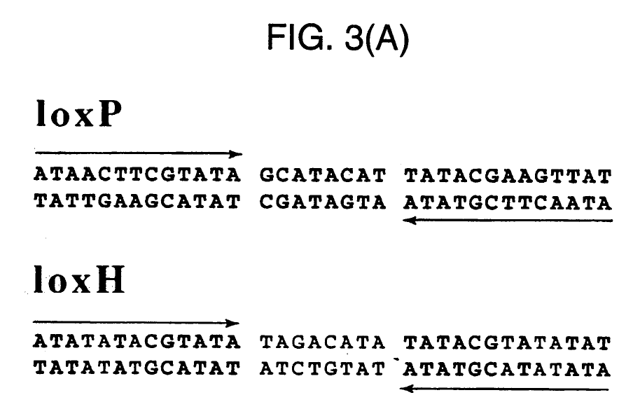

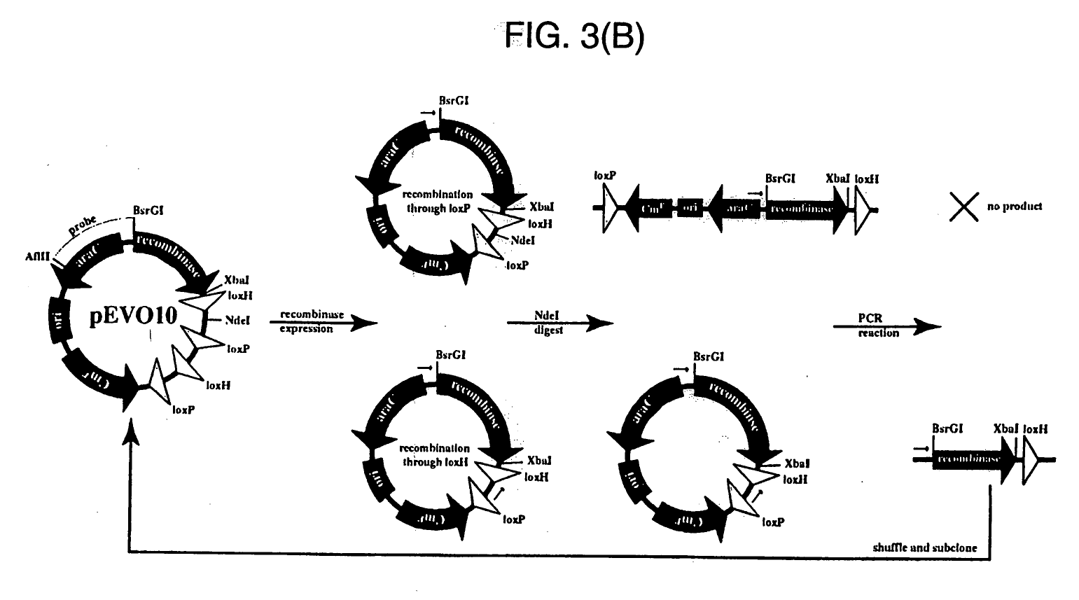

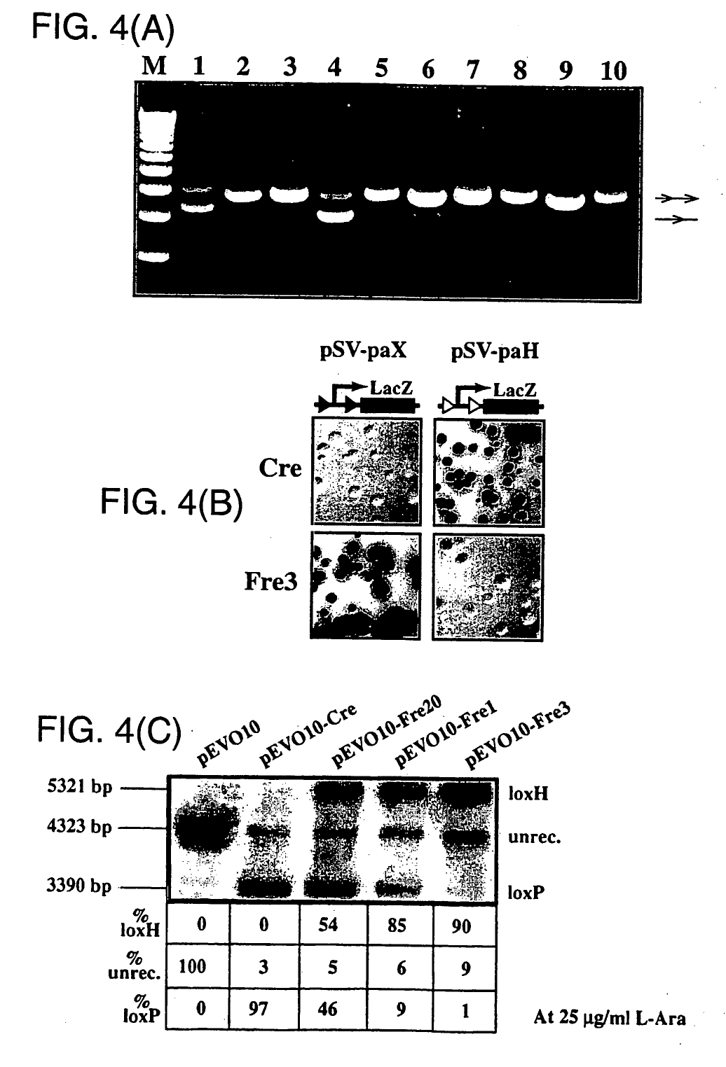

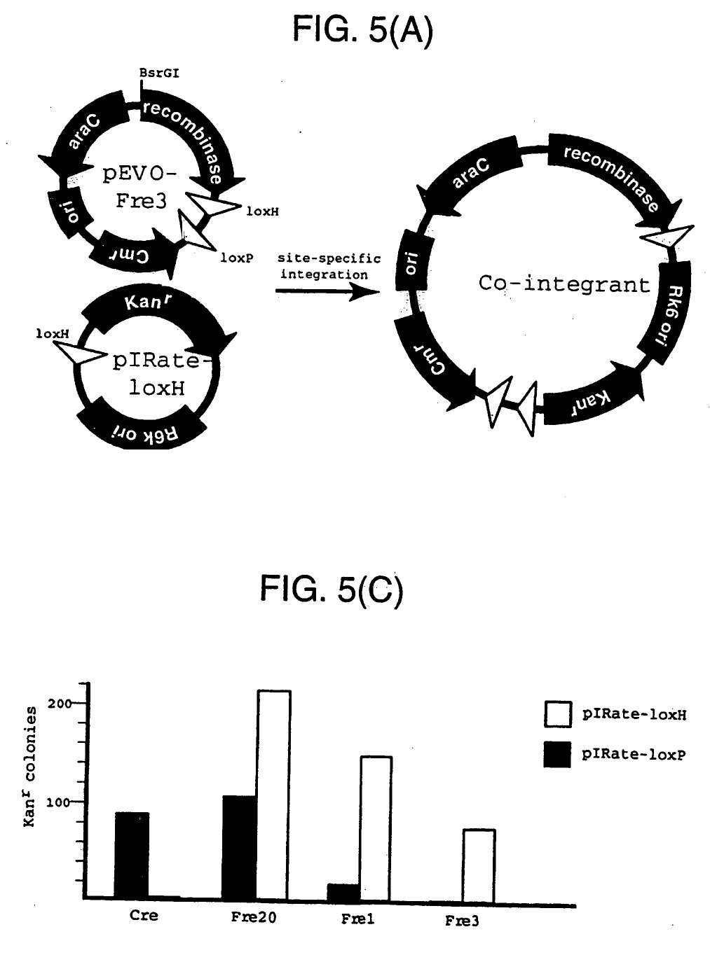

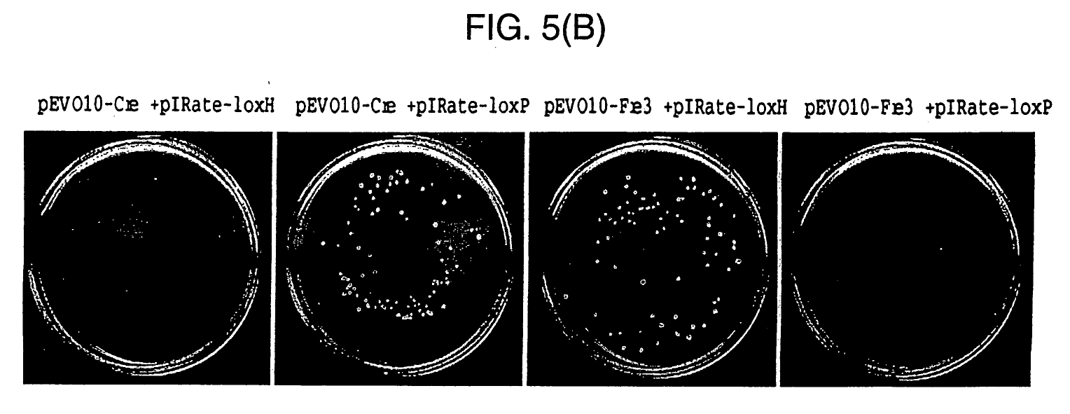

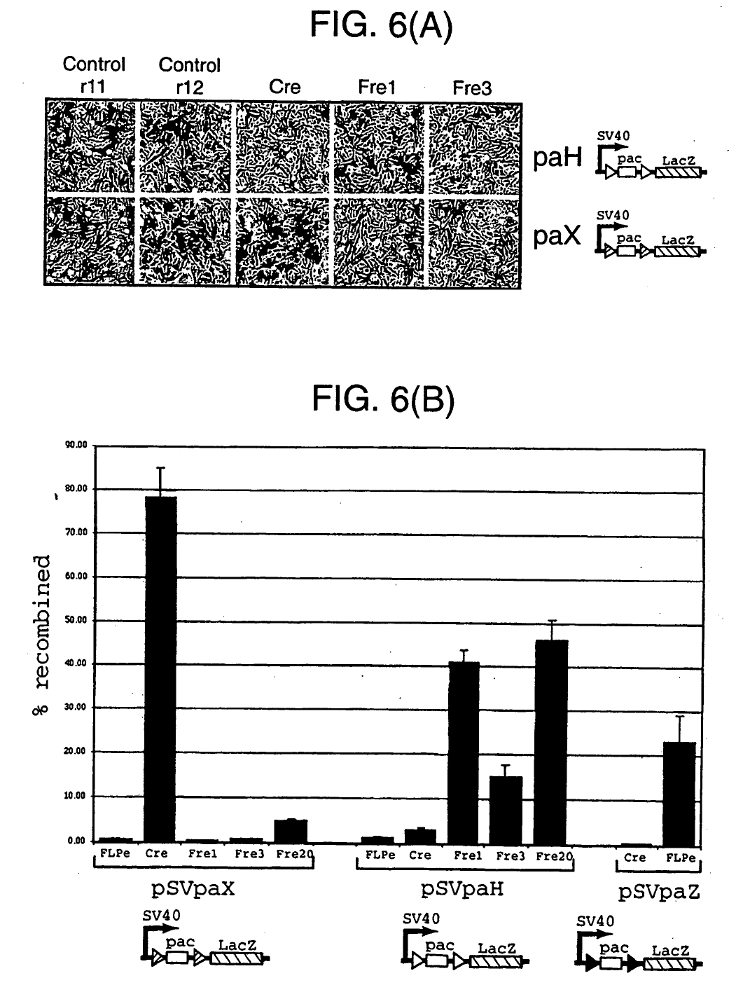

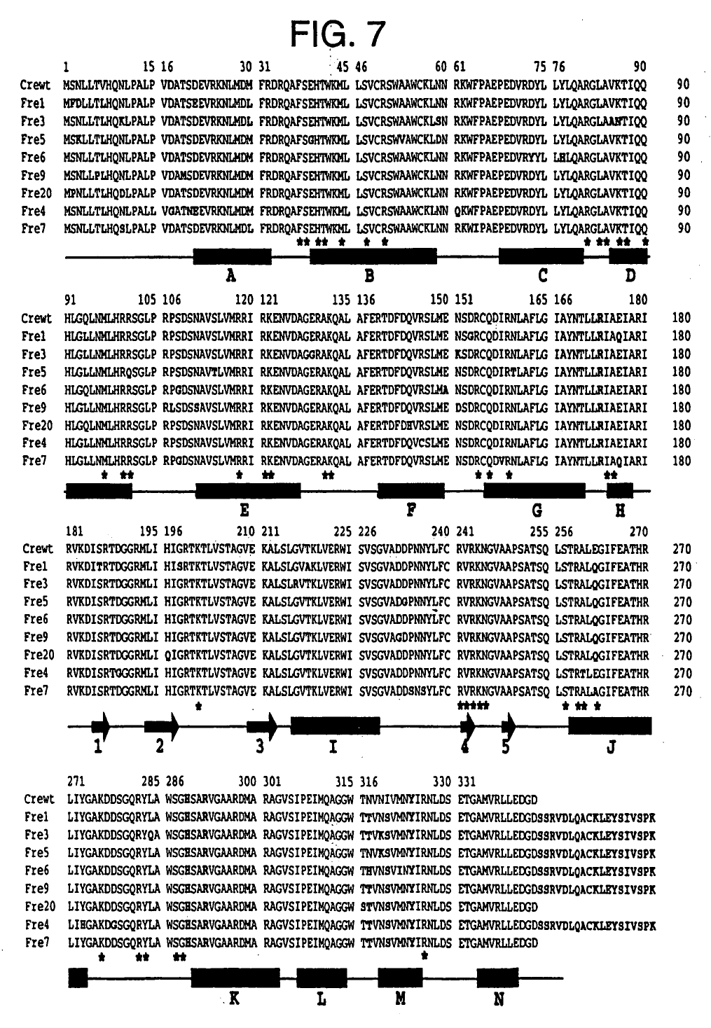

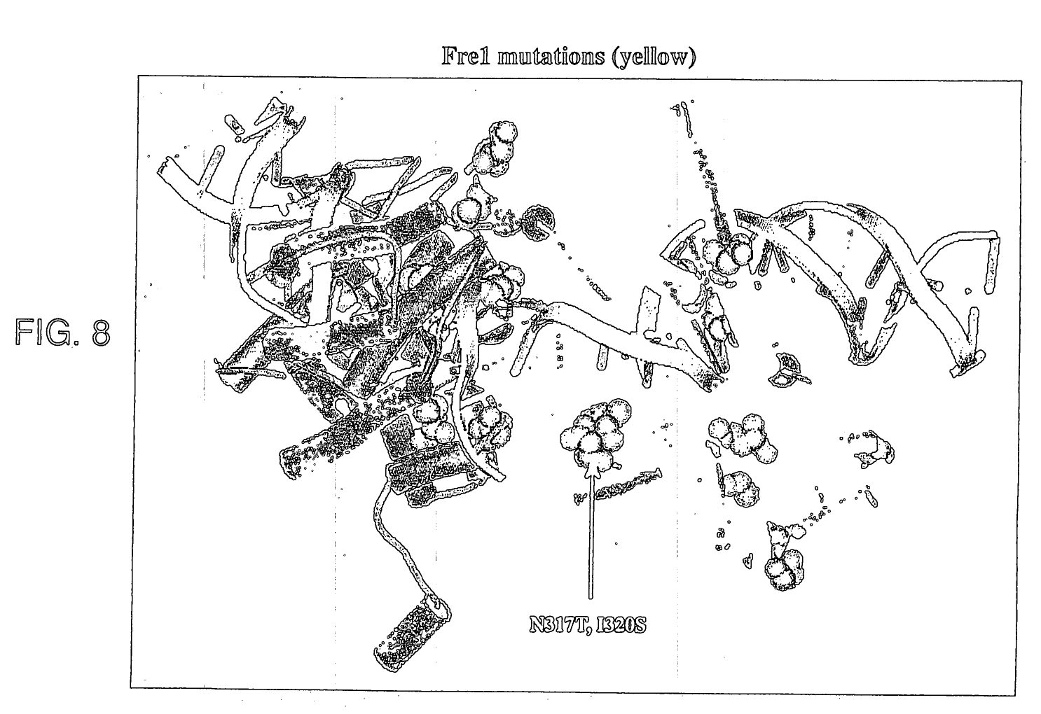

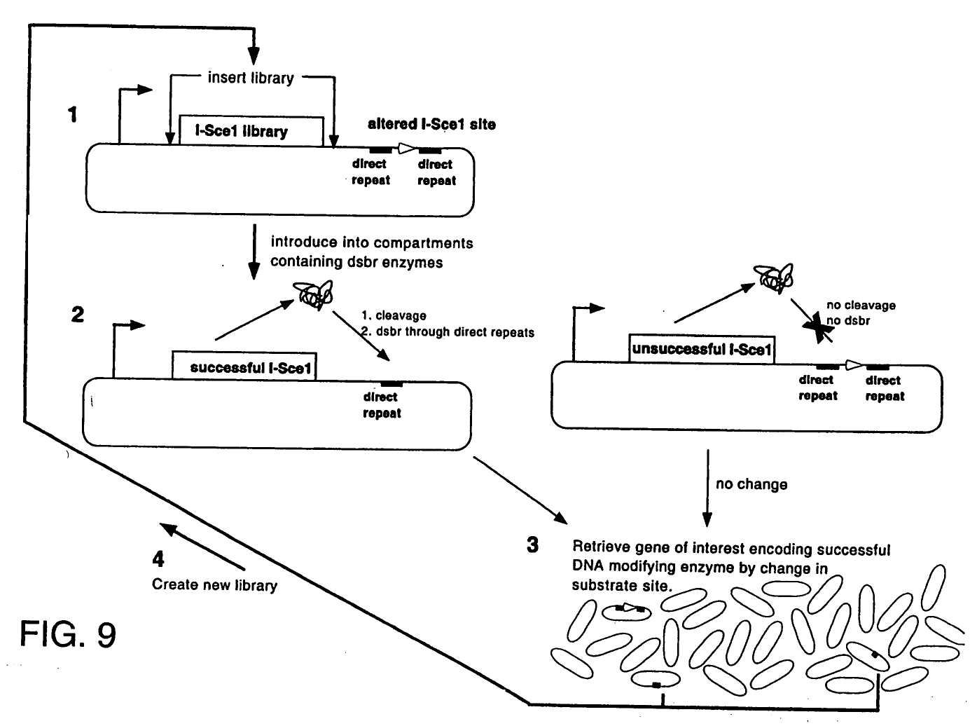

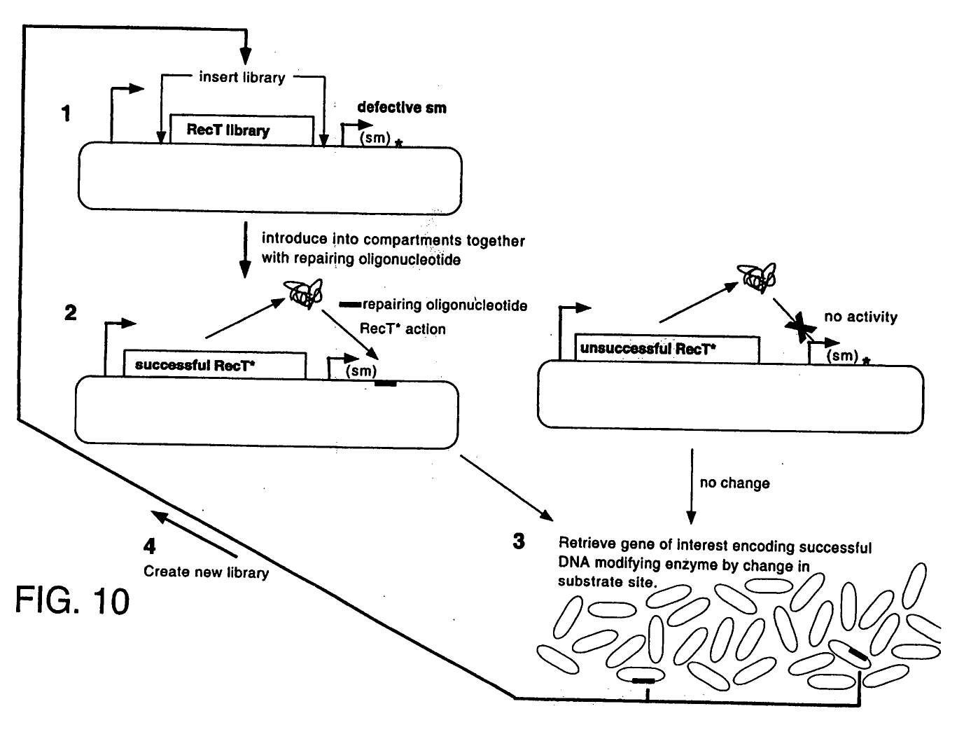

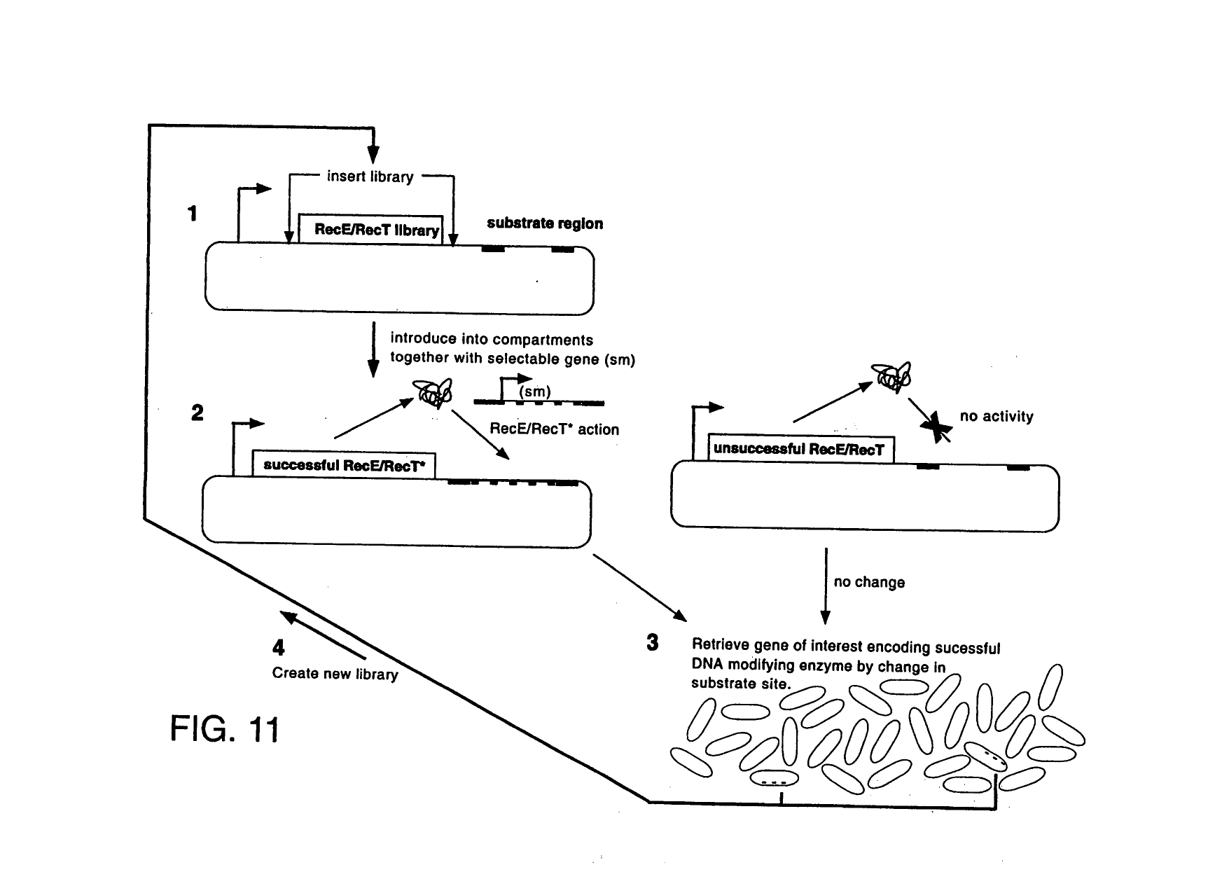

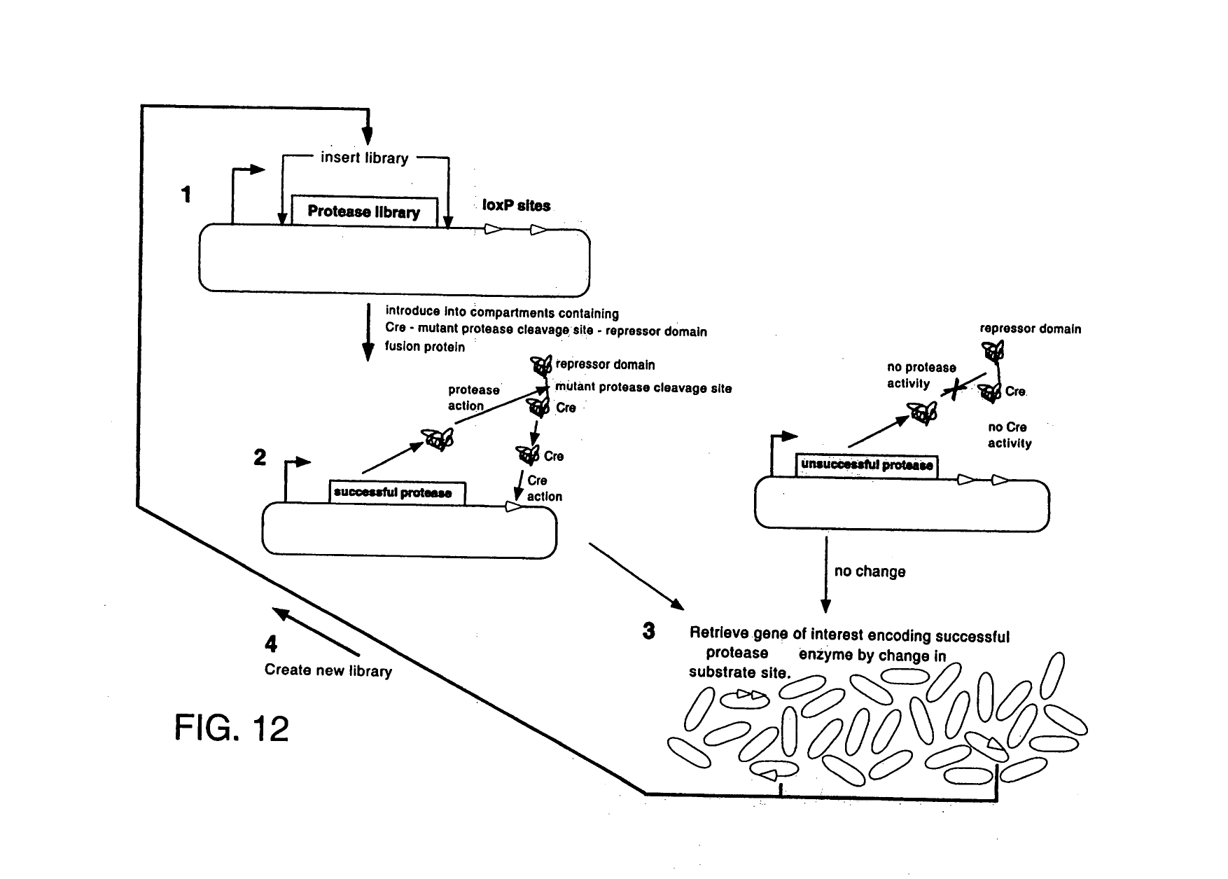

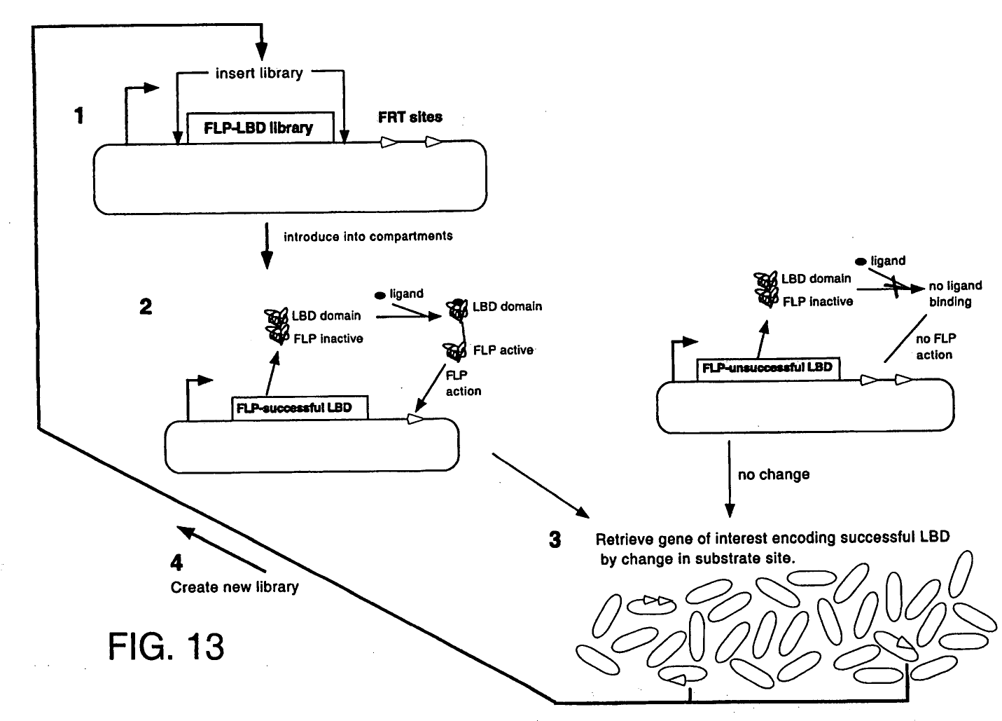

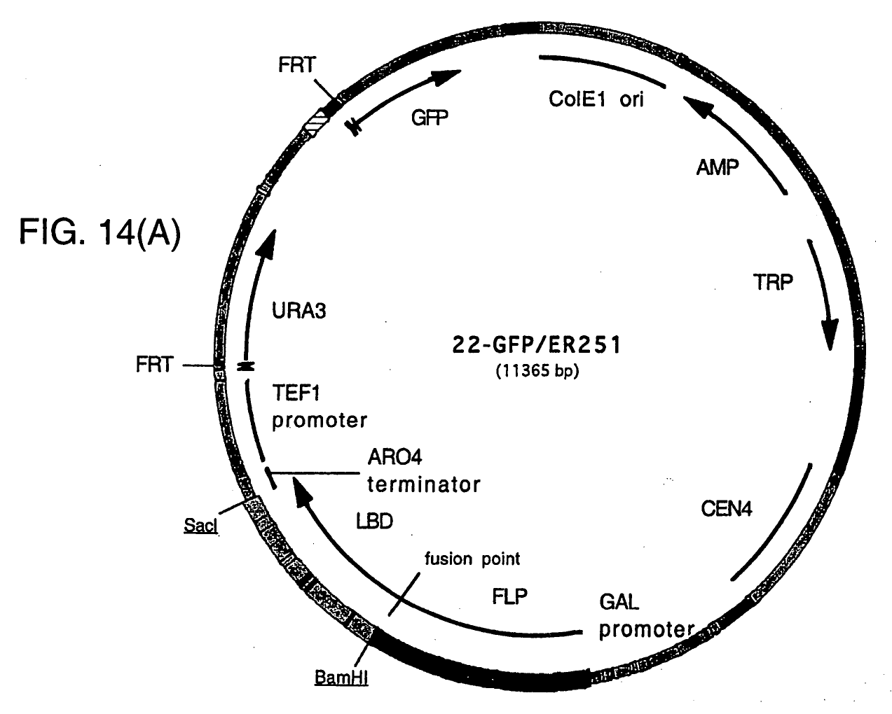

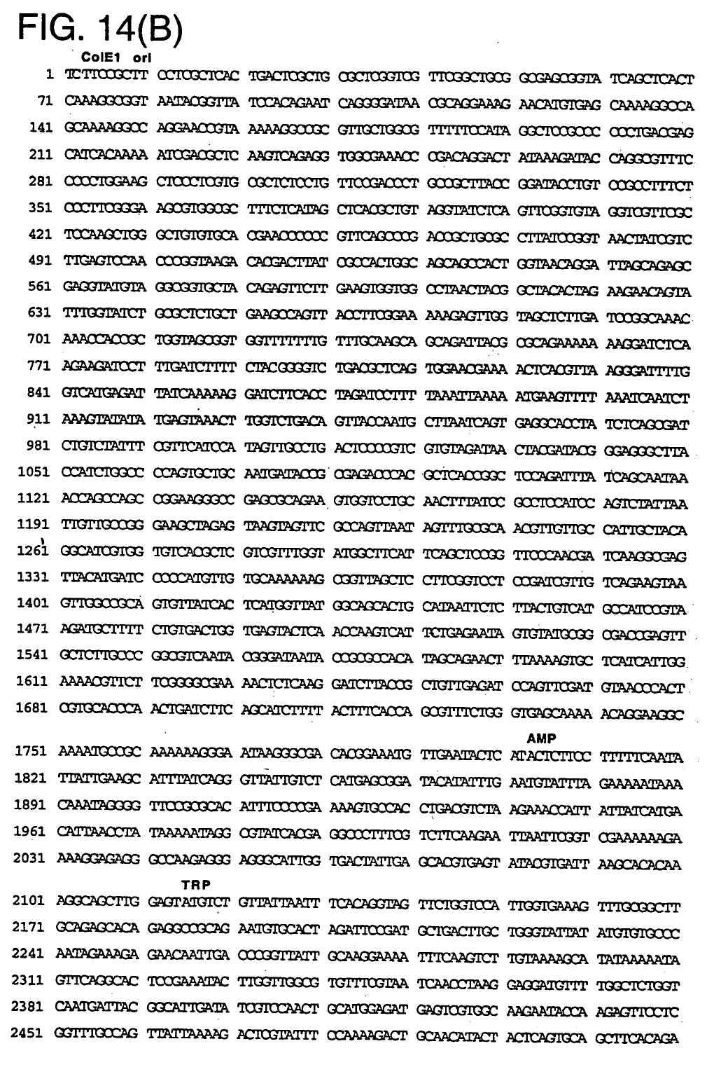

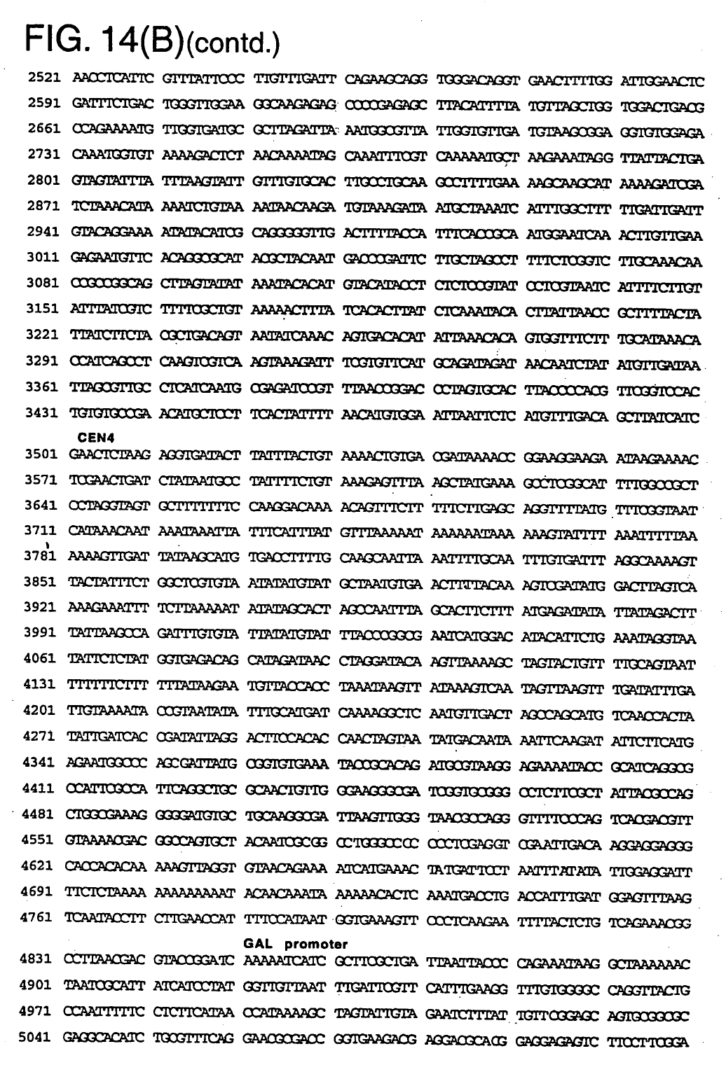

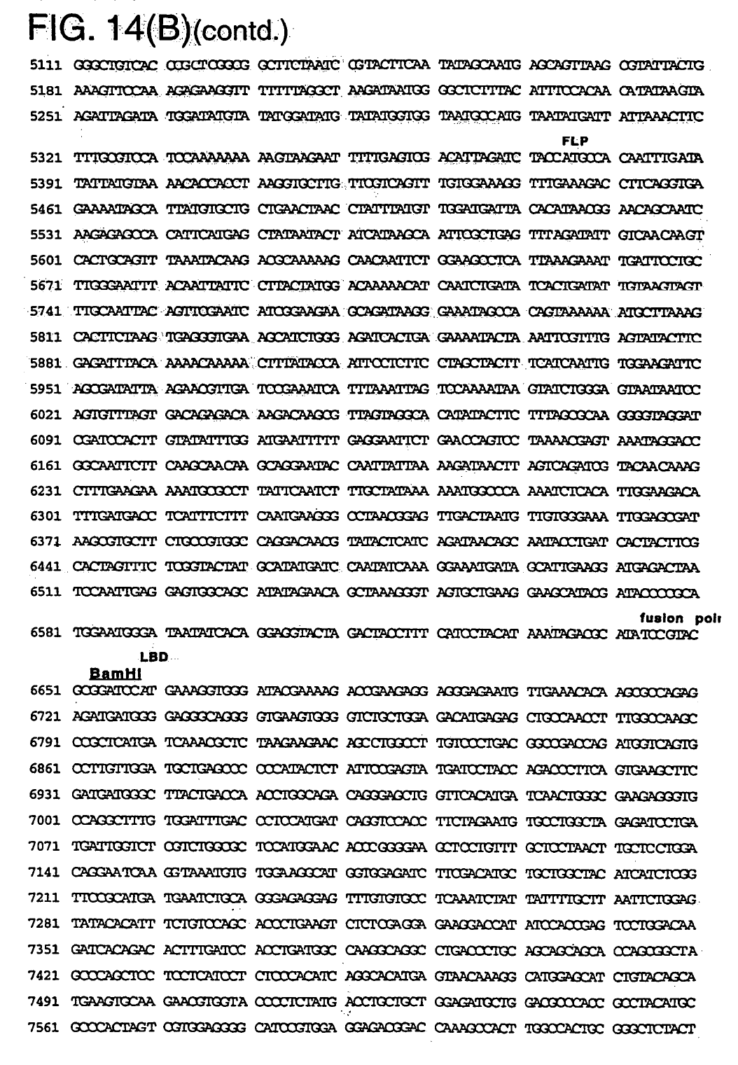

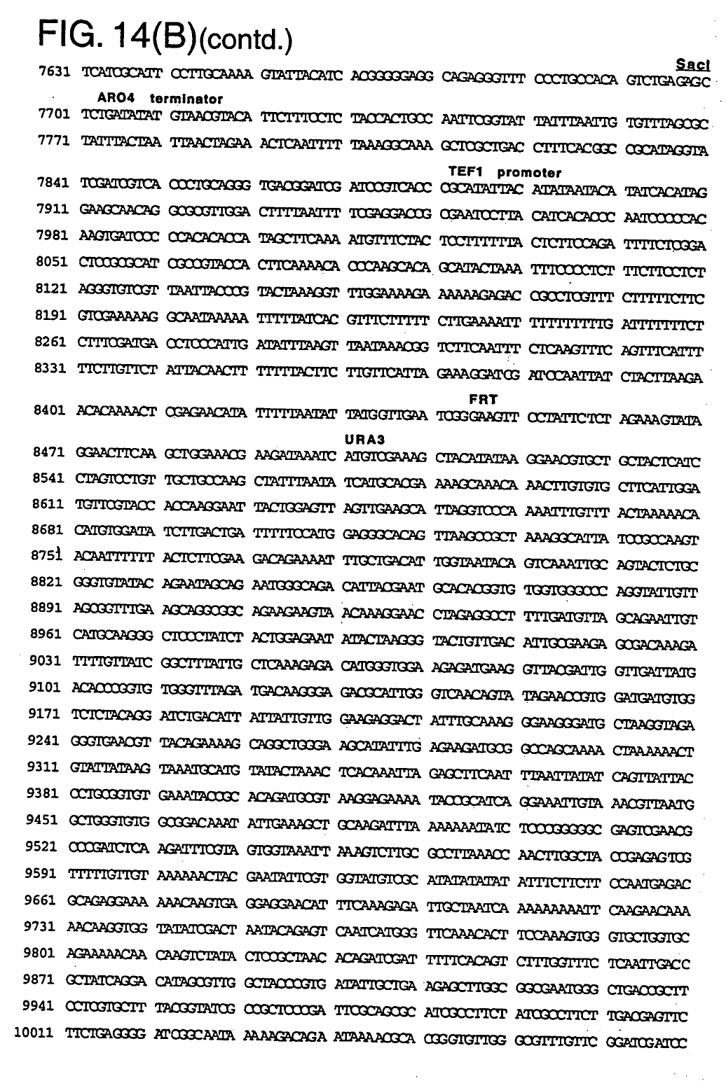

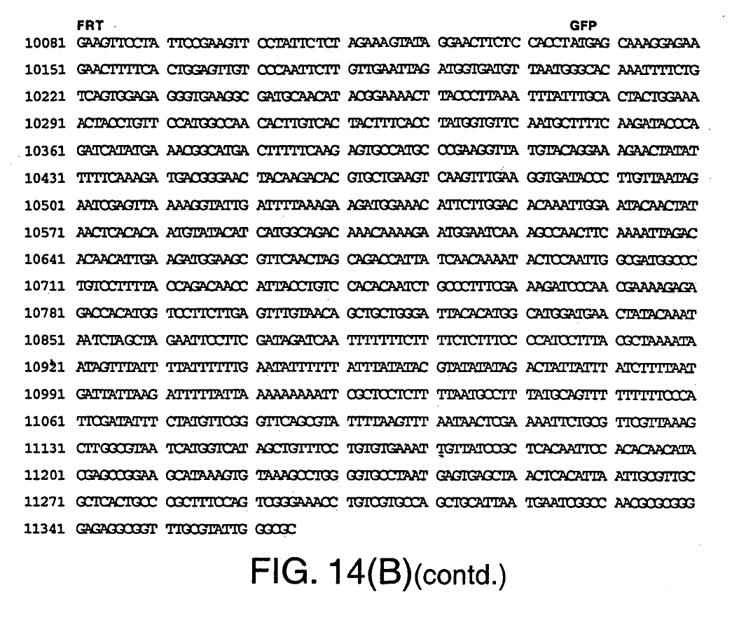

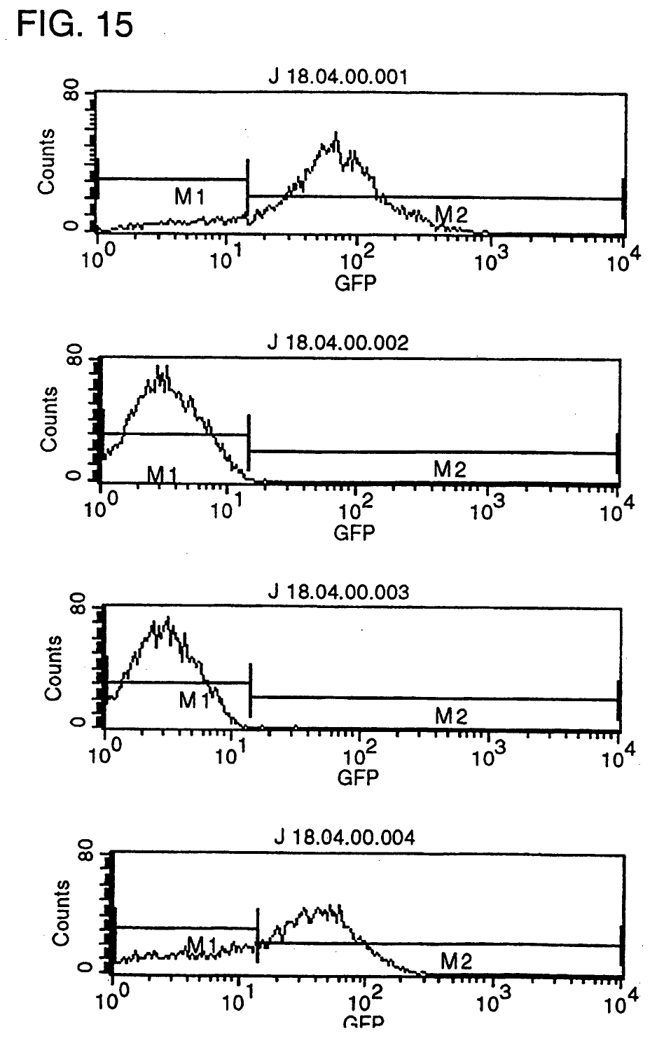

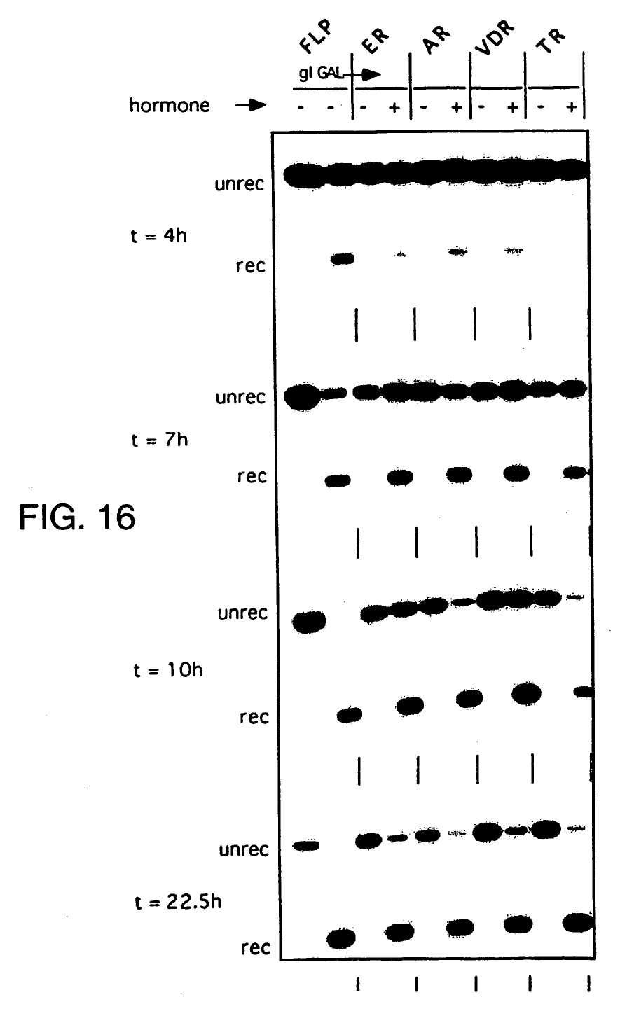

Evolution vectors: The pEVO vector series is based on the plasmid pBAD-33 (Guzman et al., J Bacteriol 177, 4121-30 (1995)). pEVO-loxP², pEVO-3, pEVO-6 and pEVO-10 are identical except for the recognition target sites for the recombinase (see also FIG. 3). pEVO-loxP² contains two tandemly repeated loxP sites as they exist in the bacteriophage P1, spaced by 690 bp. pEVO-3, contains two recognition target sites which differ in 3 nucleotides per halfsite from a loxP site. The spacer in pEVO-3 is identical to the one found in loxP sites from bacteriophage P1. pEVO-6 recognition target sites (loxH) have the spacer sequence altered in all eight positions in addition to the three nucleotides changes present in pEVO-3. pEVO-10 contains two loxH sites as well as two loxP sites, which are intertwined. Recombinase expression levels can be titrated by the amount of L-arabinose added to the medium.

Mutagenesis and DNA shuffling: Random mutations were placed into the coding sequence of Cre recombinase by error prone PCR as described (Nunes-Duby et al., Nucleic Acids Res 26, 391-406 (1998)) and by utilization of the mutator strain XL1-red (Stratagene). DNA shuffling (Stemmer, W. P. Nature 370, 389-91 (1994)) and StEP (Zhao et al., Nat Biotechnol 16, 258-61 (1998)) PCR was performed as described with minor modifications. For DNA shuffling, the whole plasmid library was segmented into 100-500 bp fragments by mild sonication and reassembled without addition of primers. The coding region of the recombinase from bacteriophage P7 was included in DNA shuffling experiments. Primers EVO-5′ (5′-TTTATCGCAACTCTCTACTG-3′) and EVO-3′ (5′-GTGTCGCCCTTATTCCCTTT[-3′) (FIG. 3) were used to amplify the reassembled coding region of the recombinase.

Generation of libraries: Amplified fragments were digested with BsrGI and XbaI and cloned into the appropriate pEVO-vector cut with the same restriction enzymes. Libraries were transfected into XL1-blue competent cells (Stratagene), transferred to liquid medium and grown in 25 μg/ml chloramphenicol and varying concentrations of L-arabinose. DNA was extracted with the Qiagen Maxi prep kit. The average library size was 1,200,000. 10 generations each were grown for the pEVO-3 and pEVO-6 series and 15 generations for pEVO-10

Breeding of recombinases: The isolated DNA from the libraries was digested with NdeI, which cuts the unrecombined (pEVO-3 and pEVO-6) and the unrecombined or loxP recombined (pEVO-10) clones, but not the plasmids that have recombined through the loxH sites. Plasmid DNA isolated from the digested library was subsequently used in a PCR reaction with 35 cycles (94° C., 1 min; 56° C., 1 min; 72° C. 1.5 min) in the presence of the primers EVO-5′ and EVO-3′. After every third generation the library was recombined by either DNA shuffling or StEP-PCR. In each generation the recombinase expression level was reduced by 20% for each vector-series, starting from 20 μg/ml L-arabinose to no L-arabinose (very low expression).

Cell culture: Chinese hamster ovary (CHO) cells were transfected with plasmid DNA using Lipofectamine (GibcoBRL). Crude cell extracts were prepared after 36 hours and Luciferase activity of cell extracts were determined with the Luciferase assay system from Promega. Relative β-galactosidase activities were measured with the Galacto-Light kit from Tropix. The Cre recombination reporter plasmid pSVpaX has been described earlier (Buchholz et al., Nucleic Acids Res 24, 4256-62 (1996)). pSVpaH is identical to pSVpaX except that the loxP sites were exchanged with loxH sites. The recombined forms of the reporter plasmids (pSVpaXΔ or pSVpaHΔ) were obtained by co-culturing pSVpaX or pSVpaH in the presence of a low copy plasmid expressing Cre or Fre3 in E.coli. Recombination efficiencies were calculated from measured β-galactosidase activities, corrected by transformation efficiencies assayed by Luciferase measurements.

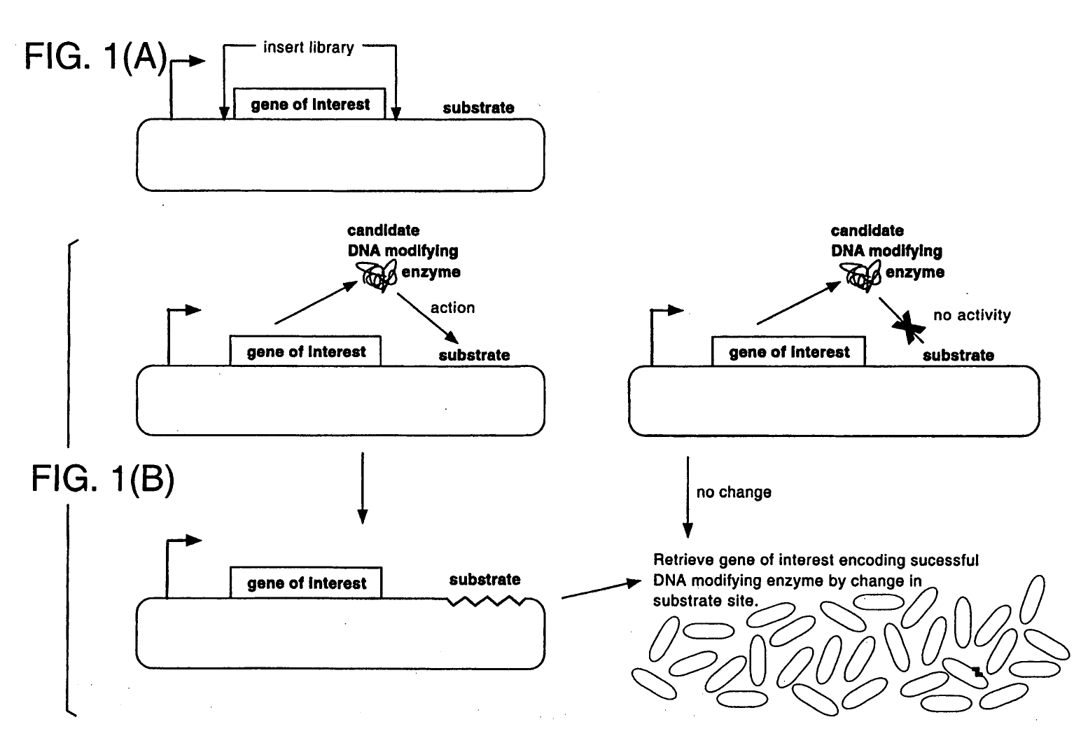

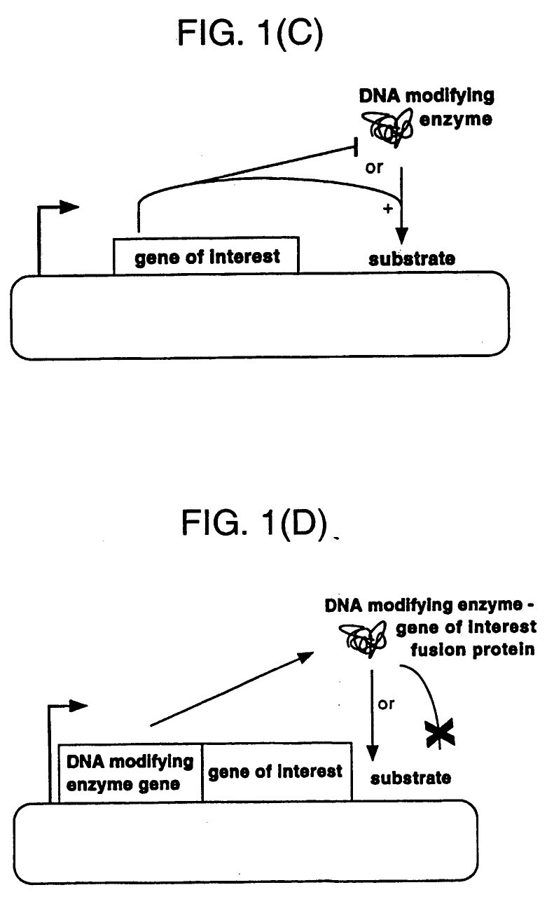

Certain schematic representations of the method of the invention are presented in FIGS. 1, 2 and 9-13.

In FIG. 1, panel A, a genetic element represented as an oval line, containing a gene of interest that can be expressed, represented as the arrowhead. This gene is physically linked to a substrate site for a DNA modifying enzyme. In many applications, the gene of interest will form part of a library of candidates.

Panel B shows a simple scheme that applies in the case where the gene of interest encodes a DNA modifying enzyme that can act upon the substrate site. When the gene of interest is a library of candidate DNA modifying enzymes, two outcomes are possible, either the candidate DNA modifying enzyme acts upon the substrate site to alter it chemically, or it does not, so leaving the substrate unchanged. The changed substrate and hence the successful candidate DNA modifying enzyme, is retrieved from a pool of genetic elements by use of the change at the substrate site. Since successful candidates are only rarely found in most molecular evolution exercises, the scheme shows the altered genetic element (wiggly line) as a rare member amongst a majority of unaltered genetic elements. The scheme shows the case where the successful event is identified because the substrate has been changed, however, the converse is also possible.

Panel C shows a simple scheme that applies in the case where the gene of interest encodes a protein that influences the activity of a DNA modifying enzyme that can act upon the substrate site. Here the gene of interest (or library of interest) does not encode a DNA modifying enzyme, but encodes molecules that regulate the DNA modifying enzyme, either to enhance (+) or to inhibit its activity. Hence the change, or lack of change, in the substrate reflects the activity of the product of the gene of interest.

Panel D shows a simple scheme that applies in the case where the coding region of the gene of interest is fused to the coding region of a DNA modifying enzyme that can act upon the substrate site. Here the gene for the DNA modifying enzyme and the gene of interest are fused so that the expressed product is a fusion between the DNA modifying enzyme and the gene of interest (or library of interest). Thus the effect of the gene of interest on the DNA modifying protein can be an intramolecular effect.

### Example 1

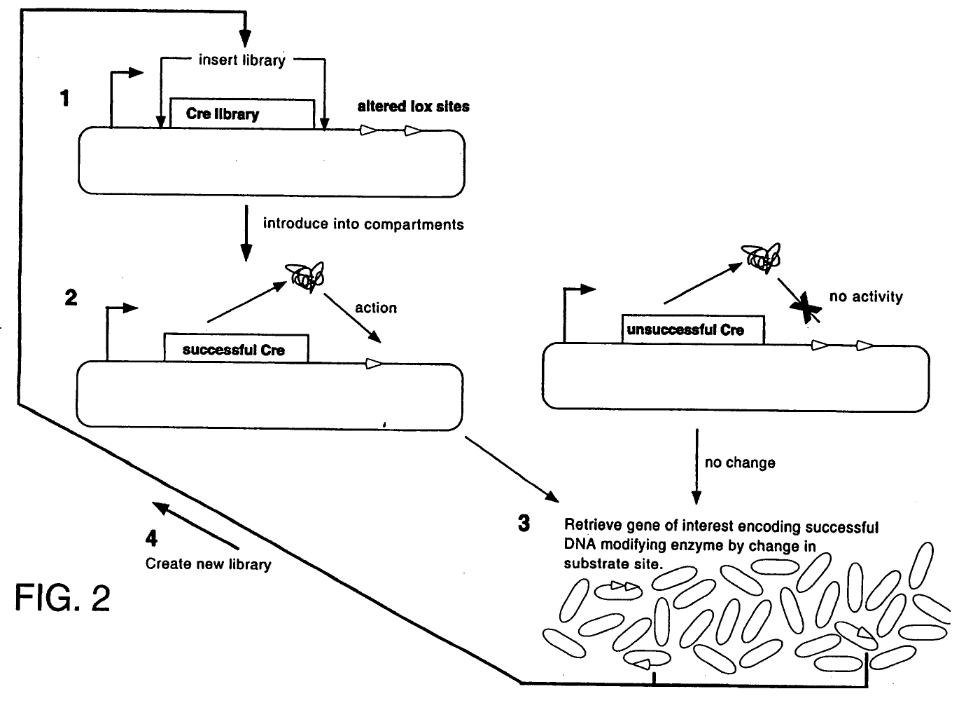

In FIG. 2, an example is presented of altering the DNA sequence specificity of a site specific recombinase. Step 1. The coding region for a site specific recombinase, in this case Cre recombinase, is mutated to create a library which is cloned into a vector that carries the intended substrate. Cre recombinase recognises a 34 bp sequence, termed loxP, and effects recombination between two loxp sites. To select for a mutant Cre recombinase that recombines between 34 bp sequences that do not represent the exact loxP consensus site, altered lox sites (represented by open triangles) are incorporated into the vector in which the mutant Cre library is cloned.

Step 2. The library is then introduced into compartments, preferably E.coli cells, in which the mutated Cre recombinases are expressed and where each member of the library is compartmentalised from all the other members of the library. Those mutants which recognise and recombine the altered lox sites change the DNA sequence proximal to their coding regions by recombination between the two altered lox sites. (Here shown as a deletion of the DNA region between the two lox sites, however strategies that employ inversion of the DNA region between two sites, or insertion of DNA into a single site, or translocation of DNA between a single site and a site present in another molecule, are also possible. In each case, the activity of a successful mutant Cre will be marked by a change in the DNA sequence that is physically linked to its coding region.) Wild type or unsuccessful mutant Cre recombinases will not catalyse the change and consequently the coding regions of the successful mutant Cre recombinases are marked by a linked change and can be retrieved from the library by a method, or methods, to identify the change.

Step 3. The change can be identified by the induction or ablation of a gene whose expression phenotypically alters the compartment. The phenotypic change can be identified by any means but preferably either (i) compartmental survival is altered so that those compartments with successful mutants are more abundant than unsuccessful mutant compartments, or (ii) compartments containing successful mutants can be rapidly sorted from compartments containing unsuccessful mutants. One such sorting method employs FACS (fluorescent activated cell sorting) technology.

The change can also be identified by any means to physically distinguish molecules altered or not by successful mutants. Preferably the alteration is identified by PCR to amplify the alteration and linked coding region for the successful mutant gene.

A further preferred embodiment combines identification by a phenotypic criterion with identification using a physical approach. Whereas either screening approach alone can identify a successful mutant from a large background of unsuccessful candidates, the combination permits the screening of even greater numbers of candidates. By these approaches, the major limitation in directed evolution of proteins, namely the identification of successful mutations that improve protein function directed at a given property, amongst the vast background of possibilities presented by random mutagenesis of protein coding regions, is addressed. In all examples presented herein, such as those expressed below, the aspects described in detail for this first example, apply to the others.

Step 4. A common end to each protein evolution cycle is the identification and amplification of successful genes, preferably by PCR. In the case illustrated in FIG. 2, successful mutant Cre recombinases were amplified by sloppy PCR so that the coding regions for the successful genes were contaminated with new mutant variations to create a new library for screening for further improved variations in the next round. Other methods to alter the proteins encoded by the successful mutants, for example DNA shuffling, can also be included at this step to create more complex libraries. A new library, based on the successful candidates identified in the previous round and altered by any means to introduce new mutations and combinations of mutations, is recloned into the vector containing the mutant lox sites, and the cycle is repeated.

A detailed application of this approach for Cre recombinase follows in Example 2.

### Example 2

Here, it was tested whether a recombinase could be generated that specifically recombines a sequence that occurs naturally in a genome. The human genome was scanned and a palindromic sequence was identified on chromosome 22 that differs in 14 out of the 34 base pair loxP site recognized by the Cre recombinase (FIG. 3a). Based on its human origin, this sequence has been designated a “loxH” site. Initial recombination experiments with loxH sites in E. coli showed that Cre recombinase does not recombine this site at measurable frequency (data not shown).

To test the method of the invention, Cre recombinase was first cloned into the vector pEVO-loxP², which contains two loxP sites, oriented as an excision substrate. Cre efficiently recombined the plasmid pEVO-loxP², when Cre was expressed from the arabinose promoter. Recombination was evident, even at very low expression levels, by the appearance of a faster migrating band (FIG. 4a, lane 1). Because Cre showed no recombination activity on loxh sites, a three step directed molecular evolution strategy was set up to allow gradual changes in the evolving recombinases to occur. As a first step, the three nucleotides different in the loxH halfsites were introduced (pEVO-3). Cre did not recombine this plasmid at low expression levels (FIG. 4a, lane 2). However, at higher expression levels recombination was observed (FIG. 4a, lane 4). Libraries of mutated recombinase were cloned into pEVO3 and screened at low recombinase expression levels. Clones that recombined this site were collectively amplified and rescreened or shuffled as outlined in methods. After 10 generations substantial amounts of the plasmids showed recombination at low recombinase expression levels (FIG. 4a, lane 6).

This library was used as the starting point in the second step and cloned into pEVO-6, which, in addition to the 3 nucleotide changes per halfsite, contains all 8 nucleotides of the altered spacer sequence (FIG 3a). Cre is sensitive to changes in the spacer sequence (Lee, G. & Saito, I. Gene 216, 55-65 (1998)) and showed no recombination when cloned into pEVO-6, even when the recombinase was induced with arabinose (FIG. 4a, lane 5). Recombinases that recombined the loxH sites in pEVO-6 evolved in further generations, evident by the recombined band in FIG. 4b, lane 7.

After 10 generations, 12 individual clones were investigated to evaluate their recombination behavior. All twelve clones recombined loxH sites to a varying degree. However, all twelve clones also showed similar or higher recombination efficiencies when they were cloned into pEVO-loxP², indicating that these recombinases possessed relaxed specificity (see clone Fre20 in FIG. 4c, and data not shown).

To identify recombinases that specifically recombine loxH sites, pEVO-10 was constructed. pEVO-10 contains two loxH sites that are intertwined with two loxP sites (FIG. 3b). Recombinases expressed in cells harboring pEVO-10 can either recombine the loxH sites, resulting in the removal of the NdeI restriction site, or the loxP sites, which removes the binding site for primer EVO-3′. Recombination of loxH with loxP is not possible because they contain different spacer sequences and homology is an essential prerequisite for recombination of integrase family site specific recombinases Hoess et al., Nucleic Acids Res 14, 2287-300 (1986); Nunes-Duby et al., Nucleic Acids Res 26, 391-406 (1998)).

Recombinases that preferably recombine loxH sites accumulated in each generation because of the higher representation of templates presented in the PCR amplification step. After 15 generaiions most recombinases investigated displayed a preference towards recombining loxH sites. Four recombinases displayed a strong preference towards recombining the loxH sites (FIG. 6b). One recombinase (designated Fre3) showed comnplete reversion of specificity in three assays (FIG. 4b, FIG. 4c), and exclusively recombined loxH sites.

The recombination properties of Fre1 and Fre3 were evaluated in mammalian cells, by co-transfecting chinese hamster ovary (CHO) cells with reporter and recombinase expression plasmids (FIG. 6a). Cells transfected with pSVpaX or pSVpaH alone showed with low β-galactosidase activity, whereas cells transfected with the recombined form of the reporter plasmids (pSVpaXΔ or pSVpaHΔ) display the β-galactosidase activity expected from complete recombination of all reporter plasmids. β-galactosidase activities measured from co-transfection of pSVpaX with pNPK-Cre indicated that approximately 75% of the reporter had recombined within 36 hours. In contrast, little recombination of plasmid pSVpaH was observed when co-transfected with the plasmid expressing Cre. As in the E.coli assays, Fre20 displayed relaxed specificity and recombined both pSVpaX and pSVpaH. Fre1 and Fre3 only recombined pSVpaH, indicating that these recombinases specifically recombined loxH sites in mammalian cells. Fre1 and Fre3 showed reduced activity in these assays when compared to Cre (FIG. 6b). Nevertheless, their activity was comparable, or better than the activity of the improved FLPe recombinase (Buchholz et al., Nat Biotechnol 16, 657-62 (1998)), which has recently been shown to work at high fidelity in mice (Rodriguez et al. Nat Genet 25, 139-40 (2000)). Selection for high enzyme activity was not included in our molecular breeding strategy. However, recombinases specifically recombining loxH sites at high fidelity might rapidly evolve in an assay that targets high enzyme activity.

DNA sequencing of individual clones after different generation cycles unmasked the power of evolutionary protein design approaches and showed the flow of evolution (FIG. 7). This data also led to the identification of important amino acid changes and predictions of their function. For instance, amino acid 262 was found to be mutated from E to Q in four out of ten clones sequenced after ten generations in pEVO3. The fact that this amino acid change was the most prominent change after ten generations in pEVO3, and that it was preserved in further generations (FIG. 7), predicts that this change enhances recombination efficiency of lox-sites that contain the three nucleotides changed in the halsite. Consistent with this hypothesis, mapping of E262 onto the Cre crystal structure shows that it is in close proximity to the changes in the loxH halfsite 10 (Guo et al., Nature 389, 40-6 (1997)) (FIG. 8).

Sequencing of fourteen clones after ten generations in pEVO6 and 15 generations in pEVO10 identified three prominent regions where amino acid changes clustered (FIG. 7). Amino acids E176, N317, N319, and I320 are facing the exposed nucleotides of the spacer sequence in the complexed synapse (FIG. 8). Amino acids M30i V85, K86, Q94, R101, S108, and E129 cover the top part of the non-cleaved site of the DNA around the spacer sequence in the same structure.

Based on the appearance of these amino acid changes after selection in pEVO3 and their close proximity to the spacer region, we predict that amino acid changes in these two clusters allow the recombination of the loxH spacer sequence, maybe by bringing the inserted spacer sequence into the correct conformation for cleavage. Interestingly, amino acids K86, Q94, R101, S108, N317, and I320 have been shown to be involved in positioning the loxP spacer sequence for cleavage in the pre-cleaved complex (Guo et al., Proc Natl Acad Sci U S A 96, 7143-8 (1999)). The third cluster comprises amino acids E150, N151, D153, and G216. In this cluster, changes that either result in the loss of a negative charge, or in the gain of a positive charge seem to be selected out. In addition, the N- and C- termini were among the fastest changing positions in the protein. This might suggest that these regions are not important for protein function and therefore, changes in these regions are well tolerated. However, some the most persistent changes were found in these regions (V7L and nucleotide deletions that extended the C-terminus by 2 or 16 amino acids), indicating that these regions might contain yet unidentified functions.

No explanation can at present be offered as to why Fre1, Fre3, Fre5, and Fre6 display specificity for loxH sites. No apparent cluster of amnino acid changes arose after fifteen generations when the library was moved from pEVO6 to pEVO10 (FIG. 7). Further generations in pEVO 10 and/or structural information of these recombinases might help to understand how the generation of specificity was accomplished.

### Example 3

In FIG. 9, the DNA sequence specificity of an endonuclease is altered.

In Step 1, the coding region for a site specific endonuclease, for example the rare cutting endonuclease I-Sce1, is mutated to create a library which is cloned into a vector that carries the intended substrate, here an altered I-Sce1 cleavage site (depicted as an open triangle). I-Sce1 recognises an approximately 20 bp sequence, and cleaves at this site. To select for a mutant I-Sce1 that cleaves at a new recognition site, an altered I-Sce1 recognition site or sites, is/are cloned into the vector into which the mutant I-Sce1 library is cloned. As described for Example A in FIG. 1, the library is then introduced into E.coli cells for expression and compartmentalisation and the further processing steps are also equivalent, except that endonuclease cleavage promotes homologous, rather than site specific, recombination to effect a change in the DNA molecule encoding the successful mutant I-Sce1. This homologous recombination event is promoted by mutant I-Sce1 endonuclease cleavage, and occurs through short direct DNA repeats previously placed either side of the introduced mutant I-Sce1 site (represented by thick black bars) and is mediated by the concomitant expression of proteins that promote double strand break repair, particularly RecE/RecT or Redα/Redβ so that the intended homologous recombination does not occur at significant frequencies in the absence of mutant I-Sce1 cleavage at a mutant I-Sce1 site. The direct repeats for intramolecular recombination can be as short as 8 bps, but longer repeats will deliver greater efficiencies. If these direct repeats are very long, for example 120 bps or greater, the background of intramolecular homologous recombination that occurs in the absence of mutant I-Sce1 cleavage will rise and may contaminate or occlude identification of the intended, mutant I-Sce1 cleavage-promoted, event.

Step 3. Thereby, vectors that carry mutant I-Sce1 genes that successfully cleave mutant I-Sce1 sites will differ physically from unsuccessful vectors. They can be identified by the physical methods described herein. Additionally, the phenotypic methods for discrimination described may also be included if the short direct repeats that promote homologous recombination are spaced either side of both a mutant I-Sce1 site and a gene whose expression presents a convenient phenotypic difference. Homologous recombination through the direct repeats will delete the phenotypic gene thus presenting both a phenotypic as well as a physical change to mark the successful mutant I-Sce1 gene for isolation and further cycling.

Step 4. A common end to each protein evolution cycle is the identification and amplification of successful genes, preferably by PCR. In the case illustrated in FIG. 9, successful mutant I-Sce1 recombinases were amplified by sloppy PCR so that the coding regions for the successful genes were contaminated with new mutant variations to create a new library for screening for further improved variations in the next round. Other methods to alter the proteins encoded by the successful mutants, for example DNA shuffling, can also be included at this step to create more complex libraries. A new library, based on the successful candidates identified in the previous round and altered by any means to introduce new mutations and combinations of mutations, is recloned into the vector containing the mutant lox sites, and the cycle is repeated.

### Example 4

In FIG. 10, step 1, the coding region(s) for a protein or proteins involved in DNA repair, for example the MSH2, MSH4, MSH6 or the E.coli phage proteins, RecT or Redβ, (here RecT), is mutated to create a library which is cloned into a vector that carries the intended substrate. The intended substrate could be a subtly mutated gene that, in its non-mutated form, can express a protein that presents an easily identifiable phenotypic change. For example, as shown here, the substrate may be an antibiotic resistance (denoted sm, for selectable marker), GFP or lacZ gene mutated by deletion of 1 to 4 or more bps, addition of 1 to 4 or more base pairs, or point mutated so that it expresses inactive protein.

Step 2. Restoration of an open reading frame by DNA repair to express an active protein presents a phenotypic way to identify successful candidates. The concomitant physical change introduced by DNA repair will also alter the vector so that it can be physically discriminated from unaltered vectors using, for example, PCR amplification conditions that discriminate between the altered and unaltered vector sequences. Alternatively, discrimination between altered and unaltered vector sequences by DNA repair may simply bypass restoration of expression of a phenotypic marker and rely solely on discrimination by physical methods.

In this case, the activity of the DNA repair proteins is directed to the substrate site on the vector by a DNA molecule (here denoted as “repairing oligonucleotide”) that encodes the repaired DNA sequence. By DNA repair, this sequence replaces the mutated region to alter the vector.

Step 3. Once repaired, the altered vector identifies the successful candidate genes from the mutant library which fuel the next round of library construction in Step 4 and further identification of successful candidates.

In contrast to examples 1 and 2 above, where the identification of successful mutations in a library of candidates relies on the acquistion of a property not encompassed by the original protein, the case described in example 3 relies on the identification of mutant proteins that show improved properties beyond those presented by the original protein. In this assay, the original protein, and non-deleterious mutant variations of the original protein, will also be successful. However, upon reiterative screening cycles, mutant variations that show improved efficiencies over the original protein will increasingly populate the pool of altered vector molecules used to generate the following round of library cloning and screening. Consequently, by the process of screening through reiterative rounds of successful candidate isolation, reassortment, recloning and testing, improved candidates will emerge.

### Example 5

In FIG. 11, the efficiency of proteins that mediate homologous recombination is improved.

Step 1. The coding region(s) for a protein or proteins involved in homologous recombination, for example the E.coli phage proteins, RecE, RecT, Redα, Redβ, UvsX, phage P22 proteins or the E.coli proteins, RecB, RecC, RecD, RecF, RecO, RecR, or any member of the RecA family, including RecA and eukaryotic RAD51s, or any member of the RAD52 family, or any other protein involved in homologous recombination (here shown as RecE/RecT) are mutated to create a library which is cloned into a vector that carries the intended substrate. The intended substrate could be a gene that can express a protein that presents an easily identifiable phenotypic change. For example, the substrate could be a mutant or wild-type antibiotic resistance, GFP or lacZ gene. Step 2. The action of the homologous recombination protein is directed towards the substrate by introduction of a DNA molecule (depicted by thick black dashes) that replaces the mutated region of the substrate gene so that the substrate gene is exchanged by homologous recombination through homology regions (depicted by thick black bars) to present the phenotypic change (here shown as the introduction of an “sm”—selectable marker—gene). The concomitant physical change in the substrate can also serve as the basis for physical methods to retrieve the linked, successful, homologous recombination genes. Alternatively, the substrate can be any DNA region physically linked to the cloning site of the introduced library and the successful genes are retrieved by use of a physical method only.

As for example 3 above, this approach relies on reiterative screening cycles to permit improved mutant variations to increasingly populate the pool of altered cloning vectors.

In FIG. 12, the scheme presents the case where the gene of interest is a protease, however the principle applies to any molecular mechanism which regulates the activity of a DNA modifying enzyme.

Step 1. A mutant library of a protease encoding gene, for example TEV or thrombin protease, is cloned into a vector nearby the substrate for a DNA modifying protein.

Step 2. In the case illustrated, the DNA modifying protein is a site specific recombinase (here Cre) and the substrate comprises two cognate site specific recombination target sites (here loxP sites depicted as open triangles). When the site specific recombinase is free to act, it will rearrange the vector by site specific recombination between the two cognate sites. The DNA region between the two site specific recombination target sites can include DNA elements so that a gene whose expression presents a phenotypic difference such as an antibiotic resistance gene is either not expressed until the site specific recombination event, or is expressed until the site specific recombination event ablates its expression. Expression of the site specific recombinase is configured so that it is expressed in all compartments in an inactive form. In the case illustrated, it is expressed as a fusion protein with an attached protein domain that inhibits the enzyme activity of the site specific recombinase. One such example of a fusion protein is the case of expression of a site specific recombinase fused to a ligand binding domain of a nuclear receptor. The fusion protein is designed so that candidate protease cleavage sites are included in the amino acid region that links the site specific recomrbinase to the inhibitory domain. Cleavage by a successful mutant proteasel at a candidate protease recognition site will sever the inhibitory domain from the site specific recombinase, thus freeing the recombinase to act on the substrate. Step 3. Thus successful mutant proteases can be retrieved by linkage to the physical change in the vector nearby to its coding region.

In the example illustrated in FIG. 13, the coding region for the DNA modifying enzyme, here the site specific recombinase, FLP, is fused to the gene of interest so that FLP is expressed as a fusion protein with mutated variations of the protein of interest. In the example illustrated, the gene of interest encodes the ligand binding domain (LBD) of a nuclear receptor. Step 2. Upon introduction and expression in a compartment, here preferably a compartment provided by a eukaryotic cell, the derived site specific recombinase/ligand binding domain (FLP-LBD) fusion proteins are inactive in the absence of cognate ligand binding by the ligand binding domain (Logie, C. and Stewart, A. F., PNAS, 1995). Before a cognate ligand is introduced into the compartment in which the fusion protein is expressed, the ligand binding domain represses the enzyme activity of the site specific recombinase so that no, or little, recombination of the substrate occurs. Upon ligand binding by the ligand binding domain, repression is relieved and recombination occurs. Thereby, in this example, the method of the invention can be applied to screen libraries of mutated ligand binding domains for successful mutant variations that bind a candidate ligand. The candidate ligand can be a single molecule, or could be a mixture of molecules. A successful mutant ligand binding domain/candidate ligand binding event will derepress the enzyme activity of the site specific recombinase and the substrate will be recombined. Step,3. As in all permutations that are described herein, the physical change in the substrate is linked to, and marks, the coding region of the successful mutant gene. It can be retreived from a large background of unsuccessful candidates by phenotypic or physical methods, or a combination of both, as described elsewhere in this submission.

### Example 6

Application of the method (SLiDE) in Saccharomyces cerevisiae.

The plasmid, 22-GFP/ER251 is depicted in FIG. 14 with its functional components labelled. The plasmid is based on a yeast/E.coli shuttle vector and consequently includes the ColE1 origin (ColE1 ori) and ampicillin resistance gene (AMP) for propagation in E.coli and the CEN4 replication origin (CEN4) and tryptophan biosynthesis gene (TRP) for propagation in yeast.

The DNA modifying protein for use in this application of SLiDE is FLP recombinase (FLP) which is expressed from the GAL promoter as a fusion protein with a ligand binding domain (LBD) from a nuclear hormone receptor.

In this scheme, and following sequence (B), the LBD is derived from the human estrogen receptor, which is fused to the very C-terminus of FLP starting at amino acid 251 of the human estrogen receptor. The fusion point is indicated between these protein encoding regions. In other derivatives of this plasmid, the unique BamH1 and Sac1 sites (indicated) are used to exchange estrogen receptor sequences for LBD sequences from other nuclear hormone receptors, or to remove any LBD so that FLP is not expressed as a fusion protein, to create 22-GFP/FLP. The FLP-LBD fusion coding region is followed by the ARO4 terminator, as indicated.

The substrate for FLP recombination includes the URA3 gene expressed from the TEF1 promoter. The URA3 gene is flanked by two FLP recombination target sites (FRTs, as indicated). Recombination mediated by FLP deletes the DNA region between the two FRTs, thereby deleting the URA3 gene.

Downstream is the gene for green fluorescent protein (GFP). Before recombination, GFP is not expressed since it has no promoter. After recombination, the GFP gene is adjacent to the TEF1 promoter and is expressed. Therefore, in this SLiDE substrate, a successful FLP recombination event results in both a physical change to the substrate plasmid adjacent to the coding region of the DNA modifying enzyme (here FLP-LBD) and also, changes in phenotypic marker gene expression (here the loss of URA3 and/or gain of GFP expression).

As shown before (Nichols, M., Rientjes, J. M. J., Logie, C. and Stewart, A. F. (1997) “Flp recombinase/estrogen receptor fusion proteins require the receptor D domain for responsiveness to antagonists, but not agonists” Mol. Endocrinol. 11, 950-961.), and diagrammed in FIG. 13, the presence of an LBD fused to FLP inhibits FLP recombinase activity and inhibition can be relieved by administering a ligand cognate for the LBD (see also FIG. 16).

The DNA sequence for plasmid 22-GFP/ER251 is presented in FIG. 14B.

Control experiments were performed with 22-GFP/FLP to establish that FLP recombination induces GFP expression, which can be then be used in FACS (fluorescent activated cell sorting) as a first, phenotypic screen for SLiDE.

FIG. 15 shows four panels. At the top, yeast cells harbouring a derivative of 22-GFP/FLP, in which the region between the two FRTs had been deleted before introduction into yeast for this experiment, is shown as a positive control for maximum GFP expression.

In the second panel, yeast cells harbouring aderivative of 22-GFP/FLP, which still carried the entire region between the FRTs but no FLP recombinase gene, is shown as a control for the absence of GFP expression.

In the third panel, yeast cells harbouring 22-GFP/FLP were cultured in glucose media, so that the GAL promoter is repressed and no FLP recombinase should be expressed. As expected, no GFP expression, indicative of a lack of FLP recombination, was observed.

In the fourth panel, yeast cells harbouring 22-GFP/FLP were cultured in galactose media to induce the GAL promoter, and hence FLP recombinase expression. As expected, GFP expression was induced, indicative of FLP recombination.

Consequently, gating a FACS sort at the M1/M2 boundary as indicated in the four panels, will separate GFP expressing from non-expressing yeast cells, and therefore those cells with active FLP recombinase those with inactive. Hence this sort can serve as a first, phenotypic criterion for molecular evolution by SLiDE.

A variety of nuclear receptor LBDs were tested in yeast for repression of FLP. The results of these experiments are shown in the form of Southern blots in FIG. 16. All LBDs tested were fused to FLP as described for 22-GFP/ER251. The LBDs tested were; ER (ER251, as above); AR (LBD of the human androgen receptor); VDR (LBD of the human vitamin D receptor) and TR (LBD of the human thyroid hormone receptor). Additionally, FLP without an attached LBD was also tested (lanes FLP).

These proteins were expressed from the GAL promoter as in 22-GFP/ER251 and were cultured either in glucose to repress expression (first lane only as indicate by ‘gl’ for FLP) or galactose to induce expression (all other lanes). At the time of galactose addition, a cognate ligand, here indicated as ‘hormone’ was added (+) or not (−). Hormones were all added at 1 μM and were; ER (estradiol); AR (mibolerone); VDR (1alpha,25-dihydroxyvitaminD3); TR (triiodothyronine); for the time periods indicated at the left, before harvesting the cells, purifying DNA and performing the Southern blots shown to examine the FLP recombination event.

Before recombination, the DNA band is larger (unrec) and recombination deletes the URA3 gene and shortens the DNA band (rec). As can be seen, in cells harbouring the FLP gene without an additional LBD, no FLP recombination is evident in cells grown in glucose (lane 1) but recombination is virtually complete within 10 hours of galactose induction (lanes 2, 11 and 12). In all FLP-LBD cases, very little recombination is evident, even at the 22.5 hour time point, in the absence of an added ligand.

In all FLP-LBD cases, recombination was efficiently induced by adding a cognate ligand. This demonstrates that the FLP-LBD proteins are expressed and the lack of recombination in the absence of a cognate ligand is due to repression by the fused LBD. Hence FLP-LBD fusion proteins clearly present suitable starting points for the SLiDE strategy outlined in FIG. 13 and developed in 22-GFP/ER251.

1. A method of selecting a nucleic acid encoding a DNA-modifying protein with a desired activity against a nucleic acid substrate comprising the steps of:
a) providing a library of genetic elements in which each genetic element includes:
i) a nucleic acid sequence encoding a DNA-modifying protein, and
ii) said nucleic acid substrate;
b) incubating said library under conditions suitable for the expression and activity of its DNA modifying proteins; and
c) selecting a nucleic acid that encodes a DNA-modifying protein with the desired activity by identifying a genetic element in which the nucleic acid substrate either has, or has not been modified.

2. A method of selecting one or more genetic elements encoding a candidate molecule having a desired activity, or having the ability to direct the synthesis of a candidate molecule having a desired activity, said method comprising the steps of:
a) providing a library of genetic elements, in which each genetic element includes:
i) a nucleic acid sequence encoding a candidate molecule for possession of the desired biological activity, or having the ability to direct the synthesis of a candidate molecule having a desired activity, and
ii) a nucleic acid sequence which constitutes a substrate for a DNA-modifying protein;
iii) a protein with DNA-modifying activity;
wherein the activity of said DNA-modifying protein is regulated by the activity of said candidate molecule, such that modification of the nucleic acid substrate only occurs in the event that the nucleic acid sequence encodes or directs the synthesis of a candidate molecule having the desired activity;
b) incubating said library and said protein with DNA-modifying activity under conditions that are suitable for its DNA-modifying activity; and
c) selecting a nucleic acid that encodes a candidate molecule with the desired activity by identifying a genetic element in which the nucleic acid substrate either has, or has not been modified.

3. A method of selecting for a nucleic acid encoding a receptor molecule with affinity for a target ligand, comprising the steps of:
a) providing a library of genetic elements in which each genetic element includes:
i) a nucleic acid sequence encoding a ligand;
ii) a nucleic acid sequence encoding a fusion protein comprising a DNA modifying protein fused to a candidate receptor molecule, wherein the DNA modifying activity of the protein is low in the absence of ligand binding to said receptor molecule and is induced, repressed or altered by binding of ligand to receptor; and
b) incubating said library under conditions suitable for the activity of its DNA modifying proteins; and
c) selecting a nucleic acid that encodes a receptor with the desired activity by identifying a genetic element in which the nucleic acid substrate either has, or has not been modified.

4. A method of selecting for a nucleic acid encoding a ligand with affinity for a target receptor comprising the steps of:
a) providing a library of genetic elements, in which each genetic element includes:
i) a nucleic acid sequence encoding a candidate ligand;
ii) a nucleic acid sequence encoding a fusion protein comprising a DNA modifying protein fused to the target receptor, wherein the DNA modifying activity of the protein is low in the absence of ligand binding to said receptor and is induced, repressed or altered by binding of ligand to receptor; and
b) incubating said library under conditions suitable for the activity of its DNA modifying proteins; and
c) selecting a nucleic acid that encodes a ligand with the desired activity by identifying a genetic element in which the nucleic acid substrate either has, or has not been modified.

5. A method according to any preceding claim additionally comprising the steps of:
d) mutagenising one or more genetic elements isolated in step (c) to create a second library;
e) repeating steps a) to c) in order to select for molecules of the desired activity.

6. A method according to any one of the preceding claims wherein in step c), a nucleic acid is selected whose sequence either has, or has not been modified.

7. A method according to any one of the preceding claims, wherein said DNA modifying protein is a recombinase protein or is a component of a recombinase complex.

8. A method according to claim 7, wherein said recombinase is a site-specific recombinase (SSR).

9. A method according to claim 8, wherein said recombinase is selected from integrase or resolvase/invertase class recombinases, and intron-encoded endonucleases, for example, FLP recombinase, Cre recombinase, R recombinase from Zygosaccharomyces rouxii plasmid pSR1, A recombinase from the Kluyveromyces drosophilarium plasmid pKD1, recombinase from the Kluyveromyces waltii plasmid pKW1, any component of the λ Int recombination system, or any component of the Gin recombination system.

10. A method according to claim 8, wherein said recombinase is a homologous recombinase.

11. A method according to any one of claims 3-10, wherein said receptor is a nuclear receptor.

12. A method according to claim 11, wherein said nuclear receptor is a hormone receptor.

13. A method according to claim 12, wherein said hormone receptor is a steroid hormone receptor.

14. A method according to claim 13, wherein the steroid hormone receptor is a vertebrate glucocorticoid, oestrogen, progesterone or androgen receptor.

15. A method according to any one of claims 6-14, wherein the recombinase protein or component of the recombinase complex is fused to the receptor or ligand binding domain thereof by means of a genetic fusion.

16. A method according to any one of the preceding claims, wherein said substrate comprises a recombinase target site.

17. A method according to any one of claims 3-16, wherein said fusion protein comprises the flp recombinase and the ligand binding domain of the oestrogen receptor.

18. A method according to any one of the preceding claims, of which at least one step is automated.

19. A method according to any one of the preceding claims, wherein there is no covalent linkage formed between the DNA modifying protein and the nucleic acid substrate.

20. A nucleic acid molecule encoding a DNA-modifying protein isolated according to the method of any one of claims 1, or 5-19.

21. A DNA-modifying protein encoded by a nucleic acid according to claim 20.

22. A DNA modifying protein according to claim 21, which is a mutant Fre or Cre recombinase as described herein.

23. A Fre recombinase according to claim 22, which is Fre 3, 5 or 20, as described herein.

24. A genetic element encoding a candidate molecule having a desired activity, isolated according to the method of any one of claims 2, or 5-19.

25. A candidate molecule encoded by a nucleic acid according to claim 24.

26. A nucleic acid encoding a receptor with affinity for a ligand, isolated according to the method of any one of claims 3, or 5-19.

27. A receptor encoded by a nucleic acid according to claim 26.

28. A nucleic acid encoding a ligand with affinity for target receptor, isolated according to the method of any one of claims 4, or 5-19.

29. A ligand encoded by a nucleic acid according to claim 28.


SEQUENCE LISTING (17 sequences)

SEQ ID NO: 1 - DNA, 20 bp, Artificial Sequence
  Descripton of Artificial Sequence EVO-5′ primer
tttatcgcaactctctactg

SEQ ID NO: 2 - DNA, 21 bp, Artificial Sequence
  Description of Artificial Sequence EVO-3′ primer
gtgtcgcccttattccctttt

SEQ ID NO: 3 - DNA, 34 bp, Artificial Sequence
  Description of Artificial Sequence loxP forward
ataacttcgtatagcatacattatacgaagttat

SEQ ID NO: 4 - DNA, 34 bp, Artificial Sequence
  Description of Artificial Sequence loxP reverse
tattgaagcatatcgatagtaatatgcttcaata

SEQ ID NO: 5 - DNA, 34 bp, Artificial Sequence
  Description of Artificial Sequence loxH forward
atatatacgtatatagacatatatacgtatatat

SEQ ID NO: 6 - DNA, 34 bp, Artificial Sequence
  Description of Artificial Sequence loxH reverse
tatatatgcatatatctgtatatatgcatatata

SEQ ID NO: 7 - protein, 343 aa, Artificial Sequence
  Description of Artificial Sequence Crewt
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 8 - protein, 362 aa, Artificial Sequence
  Description of Artificial Sequence Fre1
MFDLLTLHQNLPALPVDATSEEVRKNLMDLFRDRQAFSEHTWKMLLSVCRSWAAWCKLNNRKWFPAEPEDVRDYLLYLQARGLAVKTIQQHLGLLNMLHRRSGLPRPSDSNAVSLVMRRIRKENVDAGERAKQALAFERTDFDQVRSLMENSGRCQDIRNLAFLGIAYNTLLRIAQIARIRVKDITRTDGGRMLIHISRTKTLVSTAGVEKALSLGVAKLVERWISVSGVADDPNNYLFCRVRKNGVAAPSATSQLSTRALQGIFEATHRLIYGAKDDSGQRYLAWSGHSARVGAARDMARAGVSIPEIMQAGGWTTVNSVMNYIRNLDSETGAMVRLLEDGDSSRVDLQACKLEYSIVSPK

SEQ ID NO: 9 - protein, 362 aa, Artificial Sequence
  Description of Artificial Sequence Fre3
MSNLLTLHQKLPALPVDATSDEVRKNLMDLFRDRQAFSEHTWKMLLSVCRSWAAWCKLSNRKWFPAEPEDVRDYLLYLQARGLAANTIQQHLGLLNMLHRRSGLPRPSDSNAVSLVMRRIRKENVDAGGRAKQALAFERTDFDQVRSLMEKSDRCQDIRNLAFLGIAYNTLLRIAEIARIRVKDISRTDGGRMLIHIGRTKTLVSTAGVEKALSLRVTKLVERWISVSGVADDPNNYLFCRVRKNGVAAPSATSQLSTRALQGIFEATHRLIYGAKDDSGQRYQAWSGHSARVGAARDMARAGVSIPEIMQAGGWTTVKSVMNYIRNLDSETGAMVRLLEDGDSSRVDLQACKLEYSIVSPK

SEQ ID NO: 10 - protein, 362 aa, Artificial Sequence
  Description of Artificial Sequence Fre5
MSKLLTLHQNLPALPVDATSDEVRKNLMDMFRDRQAFSGHTWKMLLSVCRSWVAWCKLDNRKWFPAEPEDVRDYLLYLQARGLAVKTIQQHLGLLNMLHRQSGLPRPSDSNAVTLVMRRIRKENVDAGERAKQALAFERTDFDQVRSLMENSDRCQDIRTLAFLGIAYNTLLRIAEIARIRVKDISRTDGGRMLIHIGRTKTLVSTAGVEKALSLGVTKLVERWISVSGVADGPNNYLFCRVRKNGVAAPSATSQLSTRALQGIFEATHRLIYGAKDDSGQRYLAWSGHSARVGAARDMARAGVSIPEIMQAGGWTNVKSVMNYIRNLDSETGAMVRLLEDGDSSRVDLQACKLEYSIVSPK

SEQ ID NO: 11 - protein, 362 aa, Artificial Sequence
  Description of Artificial Sequence Fre6
MSNLLTLHQNLPALPVDATSDEVRKNLMDMFRDRQAFSEHTWKMLLSVCRSWAAWCKLNNRKWFPAEPEDVRYYLLHLQARGLAVKTIQQHLGQLNMLHRRSGLPRPGDSNAVSLVMRRIRKENVDAGERAKQALAFERTDFDQVRSLMANSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALQGIFEATHRLIYGAKDDSGQRYLAWSGHSARVGAARDMARAGVSIPEIMQAGGWTHVNSVINYIRNLDSETGAMVRLLEDGDSSRVDLQACKLEYSIVSPK

SEQ ID NO: 12 - protein, 362 aa, Artificial Sequence
  Description of Artificial Sequence Fre9
MSNLLPLHQNLPALPVDAMSDEVRKNLMDMFRDRQAFSEHTWKMLLSVCRSWAAWCKLNNRKWFPAEPEDVRDYLLYLQARGLAVKTIQQHLGLLNMLHRRSGLPRLSDSSAVSLVMRRIRKENVDAGERAKQALAFERTDFDQVRSLMEDSDRCQDIRNLAFLGIAYNTLLRIAEIARIRVKDISRTDGGRMLIHIGRTKTLVSTAGVEKALSLGVTKLVERWISVSGVAGDPNNYLFCRVRKNGVAAPSATSQLSTRALQGIFEATHRLIYGAKDDSGQRYLAWSGHSARVGAARDMARAGVSIPEIMQAGGWTTVNSVMNYIRNLDSETGAMVRLLEDGDSSRVDLQACKLEYSIVSPK

SEQ ID NO: 13 - protein, 343 aa, Artificial Sequence
  Description of Artificial Sequence Fre20
MPNLLTLHQDLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDHVRSLMENSDRCQDIRNLAFLGIAYNTLLRIAEIARIRVKDISRTDGGRMLIQIGRTKTLVSTAGVEKALSLGVTKLVERWISVSGVADDPNNYLFCRVRKNGVAAPSATSQLSTRALQGIFEATHRLIYGAKDDSGQRYLAWSGHSARVGAARDMARAGVSIPEIMQAGGWSTVNSVMNYIRNLDSETGAMVRLLEDGD

SEQ ID NO: 14 - protein, 362 aa, Artificial Sequence
  Description of Artificial Sequence Fre4
MSNLLTLHQNLPALLVGATNEEVRKNLMDMFRDRQAFSEHTWKMLLSVCRSWAAWCKLNNQKWFPAEPEDVRDYLLYLQARGLAVKTIQQHLGLLNMLHRRSGLPRPSDSNAVSLVMRRIRKENVDAGERAKQALAFERTDFDQVCSLMENSDRCQDIRNLAFLGIAYNTLLRIAEIARIRVKDISRTGGGRMLIHIGRTKTLVSTAGVEKALSLGVTKLVERWISVSGVADDPNNYLFCRVRKNGVAAPSATSQLSTRTLEGIFEATHRLIHGAKDGSGQRYLAWSGHSARVGAARDMARAGVSIPEIMQAGGWTTVNSVMNYIRNLDSETGAMVRLLEDGDSSRVDLQACKLEYSIVSPK

SEQ ID NO: 15 - protein, 362 aa, Artificial Sequence; identical to SEQ ID NO: 14
  Description of Artificial Sequence Fre4

SEQ ID NO: 16 - protein, 343 aa, Artificial Sequence
  Description of Artificial Sequence Fre7
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDVRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALAGIFEATHRLIYGAKDDSGQRYLAWSGHSARVGAARDMARAGVSIPEIMQAGGWTTVNSVMNYIRNLDSETGAMVRLLEDGD

SEQ ID NO: 17 - DNA, 11365 bp, Artificial Sequence
  Description of Artificial Sequence Plasmid 22-GFP/ER251
tcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgcactagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgagagcttacattttatgttagctggtggactgacgccagaaaatgttggtgatgcgcttagattaaatggcgttattggtgttgatgtaagcggaggtgtggagacaaatggtgtaaaagactctaacaaaatagcaaatttcgtcaaaaatgctaagaaataggttattactgagtagtatttatttaagtattgtttgtgcacttgcctgcaagccttttgaaaagcaagcataaaagatcgatctaaacataaaatctgtaaaataacaagatgtaaagataatgctaaatcatttggctttttgattgattgtacaggaaaatatacatcgcagggggttgacttttaccatttcaccgcaatggaatcaaacttgttgaagagaatgttcacaggcgcatacgctacaatgacccgattcttgctagccttttctcggtcttgcaaacaaccgccggcagcttagtatataaatacacatgtacatacctctctccgtatcctcgtaatcattttcttgtatttatcgtcttttcgctgtaaaaactttatcacacttatctcaaatacacttattaaccgcttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattaattctcatgtttgacagcttatcatcgaactctaagaggtgatacttatttactgtaaaactgtgacgataaaaccggaaggaagaataagaaaactcgaactgatctataatgcctattttctgtaaagagtttaagctatgaaagcctcggcattttggccgctcctaggtagtgctttttttccaaggacaaaacagtttctttttcttgagcaggttttatgtttcggtaatcataaacaataaataaattatttcatttatgtttaaaaataaaaaataaaaaagtattttaaatttttaaaaaagttgattataagcatgtgaccttttgcaagcaattaaattttgcaatttgtgatttaggcaaaagttactatttctggctcgtgtaatatatgtatgctaatgtgaacttttacaaagtcgatatggacttagtcaaaagaaattttcttaaaaatatatagcactagccaatttagcacttctttatgagatatattatagactttattaagccagatttgtgtattatatgtatttacccggcgaatcatggacatacattctgaaataggtaatattctctatggtgagacagcatagataacctaggatacaagttaaaagctagtactgttttgcagtaatttttttcttttttataagaatgttaccacctaaataagttataaagtcaatagttaagtttgatatttgattgtaaaataccgtaatatatttgcatgatcaaaaggctcaatgttgactagccagcatgtcaaccactatattgatcaccgatattaggacttccacaccaactagtaatatgacaataaattcaagatattcttcatgagaatggcccagcgattatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgctacaatcgcggcctgggccccccctcgaggtcgaattgacaaggaggagggcaccacacaaaaagttaggtgtaacagaaaatcatgaaactatgattcctaatttatatattggaggattttctctaaaaaaaaaaaaatacaacaaataaaaaacactcaaatgacctgaccatttgatggagtttaagtcaataccttcttgaaccattttccataatggtgaaagttccctcaagaattttactctgtcagaaacggccttaacgacgtaccggatcaaaaatcatcgcttcgctgattaattaccccagaaataaggctaaaaaactaatcgcattatcatcctatggttgttaatttgattcgttcatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccttcggagggctgtcacccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggatatgtatatggtggtaatgccatgtaatatgattattaaacttctttgcgtccatccaaaaaaaaagtaagaatttttgagtcgacattagatctaccatgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttagatattgtcaacaagtcactgcagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaacaagcaggaataccaattattaaaagataacttagtcagatcgtacaacaaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatatccgtacgcggatccatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggaacacccggggaagctcctgtttgctcctaacttgctcctggacaggaatcaaggtaaatgtgtggaaggcatggtggagatcttcgacatgctgctggctacatcatctcggttccgcatgatgaatctgcagggagaggagtttgtgtgcctcaaatctattattttgcttaattctggagtatacacatttctgtccagcaccctgaagtctctcgaggagaaggaccatatccaccgagtcctggacaagatcacagacactttgatccacctgatggccaaggcaggcctgaccctgcagcagcagcaccagcggctagcccagctcctcctcatcctctcccacatcaggcacatgagtaacaaaggcatggagcatctgtacagcatgaagtgcaagaacgtggtacccctctatgacctgctgctggagatgctggacgcccaccgcctacatgcgcccactagtcgtggaggggcatccgtggaggagacggaccaaagccacttggccactgcgggctctacttcatcgcattccttgcaaaagtattacatcacgggggaggcagagggtttccctgccacagtctgagagctctgatatatgtaacgtacattctttcctctaccactgccaattcggtattatttaattgtgtttagcgctatttactaattaactagaaactcaatttttaaaggcaaagctcgctgacctttcacggccgcataggtatcgatcgtcaccctgcagggtgacggatcgatccgtcacccgcatattacatataatacatatcacataggaagcaacaggcgcgttggacttttaattttcgaggaccgcgaatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgttcattagaaaggatcgatccaattatctacttaagaacacaaaactcgagaacatatttttaatattatggttgaatcgggaagttcctattctctagaaagtataggaacttcaagctggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctgcggtgtgaaataccgcacagatgcgtaaggagaaaataccgcatcaggaaattgtaaacgttaatggctgggtgtggcggacaaatattgaaagctgcaagatttaaaaaaatatctcccgggggcgagtcgaacgcccgatctcaagatttcgtagtggtaaattaaagtcttgcgccttaaaccaacttggctaccgagagtcgtttttgttgtaaaaaactacgaatattcgtggtatgtcgcatatatatatatttcttcttccaatgagacgcagaggaaaaaacaagtgaggaggaacatttcaaagagattgctaatcaaaaaaaaattcaagaacaaaaacaaggtggtatatcgactaatacagagtcaatcatgggttcaaacacttccaaagtgggtgctggtgcagaaaaacaacaagtctatactccgctaacacagatcgatttttcacagtctttggtttctcaattgaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggcgtttgttcggatcgatccgaagttcctattccgaagttcctattctctagaaagtataggaacttctccacctatgagcaaaggagaagaacttttcactggagttgtcccaattcttgttgaattagatggtgatgttaatgggcacaaattttctgtcagtggagagggtgaaggcgatgcaacatacggaaaacttacccttaaatttatttgcactactggaaaactacctgttccatggccaacacttgtcactactttcacctatggtgttcaatgcttttcaagatacccagatcatatgaaacggcatgactttttcaagagtgccatgcccgaaggttatgtacaggaaagaactatatttttcaaagatgacgggaactacaagacacgtgctgaagtcaagtttgaaggtgatacccttgttaatagaatcgagttaaaaggtattgattttaaagaagatggaaacattcttggacacaaattggaatacaactataactcacacaatgtatacatcatggcagacaaacaaaagaatggaatcaaagccaacttcaaaattagacacaacattgaagatggaagcgttcaactagcagaccattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaataatctagctagaattccttcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgcagtttttttttcccattcgatatttctatgttcgggttcagcgtattttaagtttaataactcgaaaattctgcgttcgttaaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgc